(19) United States
(12) Patent Application Publication
(10) Pub. No.: US 2024/0279740 A1
(43) Pub. Date: Aug. 22, 2024

(54) METHYLATED MARKERS FOR ACCURATE DETECTION OF PRIMARY CENTRAL NERVOUS SYSTEM AND SYSTEMIC DIFFUSE LARGE B CELL LYMPHOMA

(71) Applicants: THE JOHNS HOPKINS UNIVERSITY, Baltimore, MD (US); RENMIN HOSPITAL OF WUHAN UNIVERSITY, Wuhan (CN)

(72) Inventors: Saraswati SUKUMAR, Columbia, MD (US); Bradley Downs, Omaha, NE (US); Wanjun Ding, Wuhan (CN); Weiping Tao, Wuhan (CN)

(21) Appl. No.: 18/004,030

(22) PCT Filed: Jul. 2, 2020
(86) PCT No.: PCT/CN2020/099848

§ 371 Date: Dec. 30, 2022

(51) Int. Cl. C12Q 1/6886

(52) U.S. Cl. CPC C12Q 1/6886; C12Q 2600/154

(57) ABSTRACT

Novel methods for diagnosing and treating diffuse large B-cell lymphoma (DLBCL) and/or primary central nervous system diffuse large B-cell lymphoma (PCNSL) are provided herein. The present invention provides a set of methylation markers which were identified in silico and confirmed in archival and tissue samples could achieve 100% accuracy to discriminate DLBCL and/or PCNSL from other CNS neoplasms. The markers can be identified using QM-MSP and a novel simpler, faster, qMSP assay called TAM-MSP.

### STATEMENT OF GOVERNMENTAL INTEREST

This invention was made with government support under grant no. W81XWH-18-1-0482 awarded by the Department of Defense. The government has certain rights in the invention.

### BACKGROUND OF THE INVENTION

Primary central nervous system lymphoma is a rare but aggressive form of extranodal non-Hodgkin lymphoma (NHL) limited to the brain, spinal cord, leptomeninges and eyes (1). More than 95% of primary central nervous system lymphoma are of the diffuse large B-cell lymphoma subtype (hereinafter abbreviated to PCNSL) (2). With an incidence of 0.44 per 100,000, PCNSL accounts for approximately 2% of all primary central nervous system (PCNS) tumors (3). Since 2000, there has been an increase in the overall incidence of PCNSL, especially in the elderly, with a median age at diagnosis of 65 years (3). While being a rare tumor type, the prognosis of PCNSL is poor, with the 5- and 10-year survival rates for PCNSL at 29.9% and 22.2%, respectively (3).

The treatment of PCNSL is significantly different from that of other primary central nervous system malignant tumors. Most patients receive high-dose methotrexate combined with chemotherapy (4, 5) with and without radiotherapy. Although surgery does not significantly prolong survival in PCNSL patients, accurate diagnosis requires that patients undergo surgery to obtain pathological specimens. This delays initiation of chemotherapy and could affect the outcome of the disease (6, 7).

Previous studies have identified PCNSL-specific markers, but also demonstrated the inherent difficulty of the task of detecting them. For example, Yang et al. found that the expression level of miRNA-21 in plasma was significantly different between PCNSL and glioblastoma. Furthermore, the expression level of miRNA-21 in plasma positively correlated with that in cerebrospinal fluid (CSF) (8). While these studies showed the potential for miRNA-21 as a diagnostic marker for PCNSL, common central malignant tumors, other than glioblastoma, were not tested to confirm its specificity. In another study, Baraniskin et al. used CSF cytomorphology to diagnose PCNSL but lack of detectable malignant tumor cells or excessive cytolysis led to low levels of sensitivity (9). Somatic mutations could be detected by Next Generation Sequencing (NGS) in the cell free DNA of patients with PCNSL with a sensitivity of 24% and specificity of 100% (10). Following NGS of the primary tumor DNA, MYD88 L625P mutations were detected in 57% of patients with PCNSL in cell free DNA in plasma (11), and in CSF (12) by digital droplet PCR (ddPCR). Since the ddPCR-based techniques require prior NGS of a biopsy of the primary tumor, they are as invasive as the current methods used to diagnosis PCNSL.

In conclusion, more specific and sensitive markers, and new technologies are needed to replace surgery and stereotactic biopsy.

DNA methylation is an important epigenetic alteration in the process of tumor formation and progression. In recent years, DNA methylation markers have been tested extensively for tumor diagnosis, therapeutic monitoring, and prognosis of long-term outcome (10, 11). Due to the rarity of the tumors, there are few reports on searches for DNA methylation markers in PCNSL in archival tissue (12-15). Among these, searching for possible differences in characteristics of the CNS disease versus systemic, two consecutive methylome array studies by the same group reported no significant differences between PCNSL and DLBCL (12, 15), and similar alterations in both compared to normal blood cells.

DLBCL is the most prevalent subtype of B cell non-Hodgkin lymphoma and is the most common form of non-Hodgkin lymphoma among adults, with an annual incidence of 7 cases per 100,000 people per year in the US (3). With the use of the current standard-of-care treatment: rituximab combined with cyclophosphamide, doxorubicin, vincristine, and prednisolone (R-CHOP) patient prognosis is generally good. However, ˜30% of DLBCL patients have a poor prognosis and do not respond to treatment or relapse within 5 years after treatment (4, 5). Furthermore, for DLBCL patients in first complete remission (CR), there is no survival benefit associated with routine imaging (6).

Therefore, a rapid test applied on tissue or fluid obtained by noninvasive or minimally invasive means to differentiate primary central nervous system lymphoma and other central nervous system tumors, as well as diffuse large B-cell lymphoma, is an unmet need.

### SUMMARY OF THE INVENTION

The present inventors examined two independent large public 450K methylation array datasets, TCGA and GEO, using other malignant CNS tumors for comparison and methylation marker selection. About eight DNA methylation markers were identified that have the ability to distinguish PCNSL and/or systemic DLBCL from other malignant PCNS tumors. These findings were further validated when five of the eight markers in a sample set of archival FFPE samples using the Quantitative Methylation Specific PCR (QM-MSP) laboratory assay were successfully tested.

In some other aspects, the present inventors have now developed a novel assay called the Tailed Amplicon Multiplex Methylation Specific PCR (TAM-MSP) assay that is simpler and faster than the QM-MSP assay. TAM-MSP requires only one qPCR step since three genes are multiplexed and amplified in the same aliquot of DNA. It is a simplified version of QM-MSP since it uses ACTB as the internal loading control, not the unmethylated DNA of the same marker as its control. Thus, the number of qPCR reactions are reduced from 3 to 1. TAM-MSP due to its simplicity would be very effective for detecting methylated DNA in body fluids and blood. In one diagnosed with PCNSL and/or DLBCL, TAM-MSP will also be useful for monitoring the load of disease at the beginning of treatment, during and after the treatment to predict response and prognosticate outcome of the disease. In one free of disease, TAM-MSP may be used for surveillance to predict recurrence.

With both assays, the inventors were able to achieve 100% accuracy in distinguishing between PCNSL from eight other types of PCNS tumors.

Therefore, in accordance with a first embodiment, the present invention provides a method for identifying PCNSL and/or DLBCL in a biological sample comprising detecting the presence of one or more hypermethylated CpG regions in one or more genes of interest in the DNA from the sample, wherein the one or more genes of interest are selected from the group consisting of: NCOR2, KCNH7, DOCK1, cg05491001, SCG3, ZFPM2, GRIK1 and cg25567674; and identifying the CpG regions of the one or more of the genes of interest as hypermethylated by comparing the methylation level of the CpG regions of the one or more of the genes of interest in the sample to the methylation level of the CpG regions of the one or more of the genes of interest in normal or benign tissue sample.

In accordance with a second embodiment, the present invention provides a method for detecting the presence of one or more hypermethylated CpG regions of one or more of the genes of interest in a biological sample from a subject comprising: a) hybridizing nucleic acid obtained from the sample with one or more QM-MSP primers and probes specific for the CpG regions of the one or more genes of interest selected from the group consisting of: NCOR2, KCNH7, DOCK1, cg05491001, SCG3, ZFPM2, GRIK1 and cg25567674; b) performing QM-MSP on the sample from a); and c) detecting if any of the specific CpG regions of the one or more genes of interest of a) are hypermethylated compared to the level of methylation of the specific CpG regions of the one or more genes of interest in a normal or benign tissue sample.

In accordance with a third embodiment, the present invention provides a method for identifying PCNSL and/or DLBCL in a tissue from a subject comprising: a) hybridizing nucleic acid obtained from the sample of the subject with one or more QM-MSP primers and probes specific for the CpG regions of the one or more genes of interest selected from the group consisting of: NCOR2, KCNH7, DOCK1, cg05491001, SCG3, ZFPM2, GRIK1 and cg25567674; b) performing QM-MSP on the sample from a); c) detecting if any of the specific CpG regions of the one or more genes of interest of a) are hypermethylated compared to the level of methylation of the specific CpG regions of the one or more genes of interest in a normal or benign tissue sample; and d) identifying the subject as having PCNSL and/or DLBCL.

In accordance with a fourth embodiment, the present invention provides a method for treating PCNSL and/or DLBCL in a subject in need thereof comprising: a) hybridizing nucleic acid obtained from the sample of the subject with one or more QM-MSP primers and probes specific for the CpG regions of the one or more genes of interest selected from the group consisting of: NCOR2, KCNH7, DOCK1, cg05491001, SCG3, ZFPM2, GRIK1 and cg25567674; b) performing QM-MSP on the sample from a); c) detecting if any of the specific CpG regions of the one or more genes of interest of a) are hypermethylated compared to the level of methylation of the specific CpG regions of the one or more genes of interest in a normal or benign tissue sample; d) identifying the subject as having PCNSL and/or DLBCL and initiating surgery and adjunct radiotherapy and/or chemotherapy to the subject.

In accordance with a fifth embodiment, the present invention provides a method for detecting the presence of one or more hypermethylated CpG regions of one or more of the genes of interest in a biological sample from a subject comprising: a) hybridizing nucleic acid obtained from the sample with one or more TAM-MSP primers and probes specific for the CpG regions of a control gene and one or more TAM-MSP primers and probes specific for the CpG regions of one or more of the genes of interest selected from the group consisting of: NCOR2, KCNH7, DOCK1, cg05491001, SCG3, ZFPM2, GRIK1 and cg25567674; b) performing TAM-MSP on the sample from a); and c) detecting if any of the specific CpG regions of the one or more genes of interest of a) are hypermethylated compared to the level of methylation of the specific CpG regions of the one or more genes of interest in a normal or benign tissue sample.

In accordance with a sixth embodiment, the present invention provides a method for identifying PCNSL and/or DLBCL in a tissue from a subject comprising: a) hybridizing nucleic acid obtained from the sample with one or more TAM-MSP primers and probes specific for the CpG regions of a control gene and one or more TAM-MSP primers and probes specific for the CpG regions of one or more of the genes of interest selected from the group consisting of: NCOR2, KCNH7, DOCK1, cg05491001, SCG3, ZFPM2, GRIK1 and cg25567674; b) performing QM-MSP on the sample from a); c) detecting if any of the specific CpG regions of the one or more genes of interest of a) are hypermethylated compared to the level of methylation of the specific CpG regions of the one or more genes of interest in a normal or benign tissue sample; and d) identifying the subject as having PCNSL and/or DLBCL.

In accordance with a seventh embodiment, the present invention provides a method for identifying PCNSL and/or DLBCL in a tissue from a subject comprising: a) hybridizing nucleic acid obtained from the sample with one or more TAM-MSP primers and probes specific for the CpG regions of a control gene and one or more TAM-MSP primers and probes specific for the CpG regions of one or more of the genes of interest selected from the group consisting of: NCOR2, KCNH7, DOCK1, cg05491001, SCG3, ZFPM2, GRIK1 and cg25567674; b) performing QM-MSP on the sample from a); c) detecting if any of the specific CpG regions of the one or more genes of interest of a) are hypermethylated compared to the level of methylation of the specific CpG regions of the one or more genes of interest in a normal or benign tissue sample; and d) identifying the subject as having PCNSL and/or DLBCL, and initiating surgery and adjunct radiotherapy and/or chemotherapy to the subject.

### BRIEF DESCRIPTION OF THE DRAWINGS

FIG. 1: Characteristics of the top 8 markers identified by the Hyper-Methylation Outlier program in the marker discovery Sample dataset 1. Data from an unsupervised heatmap shows the methylation levels of the 8 identified markers found in the TCGA marker discovery sample dataset 1. All 8 markers show perfect separation of DLBC and GBMLGG in this discovery sample dataset. N=number of samples; AUC=area under the curve; CI=confidence interval; DLBC=lymphoid neoplasm diffuse large B-cell; GBMLGG=glioblastoma multiforme and brain lower grade.

FIG. 2: Performance of the 8 markers in the independent Sample dataset 2. Data from an unsupervised heatmap shows the methylation characteristics of the 8 markers in PCNSL and 11 other CNS tumors downloaded from the GEO database. N=number of samples; AUC=area under the curve; CI=confidence interval; P=Mann-Whitney statistics; PCNSL=primary central nervous system diffuse large B-cell lymphoma subtype; CNS=central nervous system.

FIGS. 3A-3B: Performance of the top two markers in the archival FFPE samples from Sample set 3. (3A) Using QM-MSP, the 5 markers, (cg054, SCG3, DOCK1, GRIK1, and KCNH7) analyzed independently, and as a two-marker panel, cg0549 and SCG3, could distinguish PCNSL (N=25) from 8 other CNS tumors (N=25) with a high level of accuracy. (3B) The histogram displays the contribution (percent methylation) of each of markers of the panel to detect PCNSL. Analysis of receiver operator characteristics (ROC, inset) show that the two-marker panel performed with a high level of accuracy with AUC of 1.00 (CI: 0.95-1.00). **=Mann-Whitney P<0.001; CMI=cumulative methylation index; N=number of samples; P=Mann-Whitney statistics; AUC=area under the curve.

FIGS. 4A-4B. Schematic illustration of the QM-MSP method and novel TAM-MSP method. (4A) The first step in the QM-MSP method uses primers to sequences without CG dinucleotides flanking the region of interest to amplify DNA. The second step in QM-MSP is nested to include several CpGs in both primer and probe locations. It quantifies both methylated and unmethylated amplicons for a single marker in a single real-time PCR reaction using 2 fluorophores. (4B) TAM-MSP method: The first step of TAM-MSP amplifies two markers and actin control (ACTB) control using a single aliquot of DNA in one well, with primers located within the CpG-rich region of interest for each methylation marker, and therefore includes CpG dinucleotides in its sequence. The 5′ end of the forward and reverse primers for the two methylation markers and ACTB have the same synthetic tails. In the second step of TAM-MSP, primers that are complimentary to the synthetic tails are used, along with marker-specific TaqMan probes, each with one of three indicated fluorescent tags. All three markers are amplified in a single real-time PCR reaction. Methylation in each marker is quantified through interpolation on a historic standard curve, and is expressed as percent cumulative methylation. Open circles: unmethylated CpG; Closed circles: methylated CpG.

FIGS. 5A-5B: TAM-MSP performance characteristics. (5A) The standard curve was made by mixing fully methylated, SssI treated human sperm DNA (HSD), and untreated unmethylated HSD at different percentages ranging from 100-3% methylation. Each dot represents the average ΔCt value of 6 replicates. (5B) Inter-assay reproducibility was calculated from the ΔCts generated from the standard curve experiment in 5A. M=methylated; N=number of replicates; CV=coefficient of variation; P=Mann-Whitney statistics.

FIGS. 6A-6B: Performance of the two markers in the Wuhan clinical sample set of archival FFPE samples using the TAM-MSP method. (6A) The two markers, cg0504 and SCG3, assessed as a panel or assessed individually, showed 100% accuracy in distinguishing PCNSL from the 8 other CNS tumors. (6B) The histogram of cumulative methylation for the two-marker panel in each sample displays the contribution of the two markers in detecting PCNSL among the CNS tumors in the sample set. Analysis of receiver operator characteristics (ROC, inset) show that the two-marker panel performed with a high degree of accuracy with AUC of 1.00 (CI: 0.95-1.00). **=Mann-Whitney P<0.001; CMI=cumulative methylation index; N=number of samples; P=Mann-Whitney statistics; AUC=area under the curve.

FIG. 7: Marker methylation in PCNSL compared to normal peripheral blood cells and brain tissues. Box plots show the methylation status as assessed from GEO datasets of PCNSL tumors compared to subpopulations of normal peripheral blood cells and normal brain tissue. Marker methylation in PCNSL, in each of the 8 markers identified in the TCGA datasets, is significantly higher than each of the subpopulations of white blood cells and normal brain tissue. N=number of samples **=P<0.001 Mann-Whitney.

FIG. 8: Spearman correlation of QM-MSP and TAM-MSP. Dot plot shows the cumulative methylation values of the top 2 markers as calculated by QM-MSP and TAM-MSP. Both show a high degree of correlation, and both methods can clearly distinguish PCNSL from other types of CNS tumors. P=Spearman correlation statistics.

FIG. 9: A table of QM-MSP Primer and probe characteristics and sequences used in the inventive methods.

FIG. 10: A table of TAM-MSP primer and probe characteristics and sequences used in the inventive methods.

### DETAILED DESCRIPTION OF THE INVENTION

PCNSL is a rare and aggressive disease that requires a brain biopsy for accurate diagnosis. The inventors theorized that carefully selected methylation markers could distinguish between PCNSL and/or systemic DLBCL from other CNS neoplasms.

Through the reverse engineering of previously identified 450K methylation markers that have shown to have reproducible utility in different qMSP assays, the inventors developed a set of heuristic rules for the HyperMethylated Outlier Detector program (10, 22, 23). Although these simple heuristic rules may not suitable to address all biological questions, the inventors now show that the markers identified by this program could be developed successfully for at least two quantitative MSP assays, QM-MSP and TAM-MSP, and retained a level of accuracy equal to, or better than those observed in the methylation arrays.

Using the algorithm, eight promising markers in the TCGA dataset were identified. These markers were tested in an independent GEO dataset of PCNSL (N=95), and other primary brain tumors (N=2,112), and validated in 25 archival PCNSL tumors, and 25 tumors from eight other types of CNS neoplasms by a novel quantitative TAM-MSP assay.

In an embodiment, two markers, SCG3 and cg054, analyzed with actin in one reaction, could achieve 100% accuracy to discriminate DLBCL and/or PCNSL from other CNS neoplasms (AUC=1.00, 95% CI=1.00-1.00, P<0.001). This systematic analysis, from discovery in silico to validation of the novel tumor-specific markers in tissue samples, is the first key step to developing a less invasive, circulating cell free DNA-based test in cerebrospinal fluid or blood for DLBCL and/or PCNSL.

As such, in accordance with a first embodiment, the present invention provides a method for identifying DLBCL and/or PCNSL in a biological sample comprising detecting the presence of one or more hypermethylated CpG regions in one or more genes of interest in the DNA from the sample, wherein the one or more genes of interest are selected from the group consisting of: NCOR2, KCNH7, DOCK1, cg05491001, SCG3, ZFPM2, GRIK1 and cg25567674; and identifying the CpG regions of the one or more of the genes of interest as hypermethylated by comparing the methylation level of the CpG regions of the one or more of the genes of interest in the sample to the methylation level of the CpG regions of the one or more of the genes of interest in normal or benign tissue sample.

In some embodiments, DLBCL and/or PCNSL can be detected in a biological sample by detecting the presence of one hypermethylated CpG region in one or more genes of interest in the DNA. In some embodiments, DLBCL and/or PCNSL can be detected in a biological sample by detecting the presence of two, three, four, five, six, or seven hypermethylated CpG regions in one or more genes of interest in the DNA.

In some embodiments, DLBCL and/or PCNSL can be detected in a biological sample by detecting the presence of one or more of the following pairs of hypermethylated CpG regions: SCG3 and DOCK1, SCG3 and cg054, SCG3 and cg255, DOCK1 and cg054, and GRIK1 and cg255. These pairs of gene regions of interest can be used with QM-MSP and/or TAM-MSP methods described herein.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In accordance with a second embodiment, the present invention provides a method for detecting the presence of one or more hypermethylated CpG regions of one or more of the genes of interest in a biological sample from a subject comprising: a) hybridizing nucleic acid obtained from the sample with one or more QM-MSP primers and probes specific for the CpG regions of the one or more genes of interest selected from the group consisting of: NCOR2, KCNH7, DOCK1, cg05491001, SCG3, ZFPM2, GRIK1 and cg25567674; b) performing QM-MSP on the sample from a); and c) detecting if any of the specific CpG regions of the one or more genes of interest of a) are hypermethylated compared to the level of methylation of the specific CpG regions of the one or more genes of interest in a normal or benign tissue sample.

In accordance with a third embodiment, the present invention provides a method for identifying DLBCL and/or PCNSL in a tissue from a subject comprising: a) hybridizing nucleic acid obtained from the sample of the subject with one or more QM-MSP primers and probes specific for the CpG regions of the one or more genes of interest selected from the group consisting of: NCOR2, KCNH7, DOCK1, cg05491001, SCG3, ZFPM2, GRIK1 and cg25567674; b) performing QM-MSP on the sample from a); c) detecting if any of the specific CpG regions of the one or more genes of interest of a) are hypermethylated compared to the level of methylation of the specific CpG regions of the one or more genes of interest in a normal or benign tissue sample; and d) identifying the subject as having DLBCL and/or PCNSL.

In accordance with a fourth embodiment, the present invention provides a method for treating DLBCL and/or PCNSL in a subject in need thereof comprising: a) hybridizing nucleic acid obtained from the sample of the subject with one or more QM-MSP primers and probes specific for the CpG regions of the one or more genes of interest selected from the group consisting of: NCOR2, KCNH7, DOCK1, cg05491001, SCG3, ZFPM2, GRIK1 and cg25567674; b) performing QM-MSP on the sample from a); c) detecting if any of the specific CpG regions of the one or more genes of interest of a) are hypermethylated compared to the level of methylation of the specific CpG regions of the one or more genes of interest in a normal or benign tissue sample; d) identifying the subject as having DLBCL and/or PCNSL and initiating surgery and adjunct radiotherapy and/or chemotherapy to the subject.

It will be understood by those of ordinary skill in the art that the inventive methods can comprise identifying at least one CpG region of at least one gene of interest from a sample of the subject as being hypermethylated compared to the same CpG region of at least one gene of interest from a normal/benign sample. For example, CpG regions in SCG3 and cg054 gene regions are useful for diagnosis and treatment using QM-MSP methods.

In accordance with a fifth embodiment, the present invention provides a method for detecting the presence of one or more hypermethylated CpG regions of one or more of the genes of interest in a biological sample from a subject comprising: a) hybridizing nucleic acid obtained from the sample with one or more TAM-MSP primers and probes specific for the CpG regions of a control gene and one or more TAM-MSP primers and probes specific for the CpG regions of one or more of the genes of interest selected from the group consisting of: NCOR2, KCNH7, DOCK1, cg05491001, SCG3, ZFPM2, GRIK1 and cg25567674; b) performing TAM-MSP on the sample from a); and c) detecting if any of the specific CpG regions of the one or more genes of interest of a) are hypermethylated compared to the level of methylation of the specific CpG regions of the one or more genes of interest in a normal or benign tissue sample.

In accordance with a sixth embodiment, the present invention provides a method for identifying DLBCL and/or PCNSL in a tissue from a subject comprising: a) hybridizing nucleic acid obtained from the sample with one or more TAM-MSP primers and probes specific for the CpG regions of a control gene and one or more TAM-MSP primers and probes specific for the CpG regions of one or more of the genes of interest selected from the group consisting of: NCOR2, KCNH7, DOCK1, cg05491001, SCG3, ZFPM2, GRIK1 and cg25567674; b) performing QM-MSP on the sample from a); c) detecting if any of the specific CpG regions of the one or more genes of interest of a) are hypermethylated compared to the level of methylation of the specific CpG regions of the one or more genes of interest in a normal or benign tissue sample; and d) identifying the subject as having DLBCL and/or PCNSL.

In accordance with a seventh embodiment, the present invention provides a method for identifying DLBCL and/or PCNSL in a tissue from a subject comprising: a) hybridizing nucleic acid obtained from the sample with one or more TAM-MSP primers and probes specific for the CpG regions of a control gene and one or more TAM-MSP primers and probes specific for the CpG regions of one or more of the genes of interest selected from the group consisting of: NCOR2, KCNH7, DOCK1, cg05491001, SCG3, ZFPM2, GRIK1 and cg25567674; b) performing QM-MSP on the sample from a); c) detecting if any of the specific CpG regions of the one or more genes of interest of a) are hypermethylated compared to the level of methylation of the specific CpG regions of the one or more genes of interest in a normal or benign tissue sample; and d) identifying the subject as having DLBCL and/or PCNSL and initiating surgery and adjunct radiotherapy and/or chemotherapy to the subject.

It will be understood by those of ordinary skill in the art, that the detection of methylation of the CpG regions of the genes of interest and the level of methylation detected in the samples from suspicious lesions of a subject is compared to the methylation levels of the CpG regions of the genes of interest in normal tissue or benign tissue. When the level is elevated in the sample compared to normal control tissue or benign tissue, the lesion is diagnosed as having DLBCL and/or PCNSL. The particular gene panel of the inventive methods were specifically chosen to identify those genes which were very highly methylated when DLBCL and/or PCNSL was present, and had little or no methylation when in normal or benign tissue. Moreover, when the level of methylation of the genes in the sample from the sample is not significantly different that normal or benign tissue, there no risk of DLBCL and/or PCNSL in the tissue sampled.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids used as primers in embodiments of the present invention can be constructed based on chemical synthesis and/or enzymatic ligation reactions using procedures known in the art. See, for example, Sambrook et al. (eds.), Molecular Cloning. A Laboratory Manual, 3^{rd} Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxy carboxymethyluracil, 5-methoxyuracil, 2-methylthio-N⁶-isopenteny ladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

The term “isolated and purified” as used herein means a protein that is essentially free of association with other proteins or polypeptides, e.g., as a naturally occurring protein that has been separated from cellular and other contaminants by the use of antibodies or other methods or as a purification product of a recombinant host cell culture.

The term “biologically active” as used herein means an enzyme or protein having structural, regulatory, or biochemical functions of a naturally occurring molecule.

It will be understood by those of ordinary skill in the art that the methods of the present invention can be used to diagnose, prognosticate, and monitor treatment of any disease or biological state in which methylation of genes is correlative of such a disease or biological state in a subject. In some embodiments, the disease state is breast cancer. In some embodiments, the type of breast cancer can be invasive ductal carcinoma or ductal carcinoma in situ.

In accordance with one or more embodiments of the present invention, it will be understood that the types of cancer diagnosis which may be made, using the methods provided herein, is not necessarily limited. For purposes herein, the cancer is DLBCL and/or PCNSL. As used herein, the term “cancer” is meant any malignant growth or tumor caused by abnormal and uncontrolled cell division that may spread to other parts of the body through the lymphatic system or the blood stream.

The cancer can be a metastatic cancer or a non-metastatic (e.g., localized) cancer, an invasive cancer or an in situ cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of diagnosis, staging, screening, or other patient management, including treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules.

As used herein, the term “selective hybridization” or “selectively hybridize” refers to hybridization under moderately stringent or highly stringent conditions such that a nucleotide sequence associates with a selected nucleotide sequence but not with unrelated nucleotide sequences. Generally, an oligonucleotide useful as a probe or primer that selectively hybridizes to a selected nucleotide sequence is at least about 15 nucleotides in length, usually at least about 18 nucleotides, and particularly about 20 nucleotides in length or more in length. Conditions that allow for selective hybridization can be determined empirically, or can be estimated based, for example, on the relative GC: AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual” (Cold Spring Harbor Laboratory Press 1989)).

“Identical” or “identity” as used herein in the context of two or more nucleic acids or polypeptide sequences may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

As used herein the term “optically detectable DNA probe” means an oligonucleotide probe that can act as a molecular beacon or an oligonucleotide probe comprising a fluorescent moiety or other detectable label, with or without a quencher moiety.

“Substantially complementary” used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

In some embodiments, the inventive methods herein employ two-step quantitative multiplex-methylation specific PCR (QM-MSP). The methods are disclosed in U.S. Pat. Nos. 8,062,849 and 9,416,404 which are hereby incorporated herein as if set forth in their entireties. The QM-MSP technique combines the sensitivity of multiplex PCR with the quantitative features of quantitative methylation-specific PCR (Q-MSP) in such a way that a panel of genes whose hypermethylation is associated with a type of carcinoma can be co-amplified from limiting amounts of DNA derived from tissue or samples sources of the subject being tested. The invention methods also provide quantitative definition of the extent of gene hypermethylation in normal appearing tissues on a gene-by-gene basis. Thus, the inventive methods can be used to more powerfully discriminate between normal or benign tissues and malignant tissues and to monitor or assess the course of cancer development in a subject.

### Use of Quantitative Multiplex Methylation Specific PCR

QM-MSP is a highly sensitive, specific and quantitative methylation assay. It combines the principles of conventional gel-based MSP, quantitative real time MSP, and multiplexed gel-based MSP into one format developed to enable quantification of methylated gene panels in clinical samples with limited DNA quantities available. It has an analytical sensitivity of one methylated copy in 100,000 unmethylated copies, which is nearly 10 fold higher than QMSP, and 100 fold higher than gel based MSP techniques.

QM-MSP is a two-step PCR approach for a multiplexed analysis of a panel of up to 14 genes in clinical samples with minimal quantities of DNA. In the first step, the External PCR reaction, for up to 14 genes tested, one pair of gene-specific primers (forward and reverse) amplifies the methylated and unmethylated copies of the same gene simultaneously and in multiplex, in one PCR reaction. This is a methylation-independent amplification step, to increase the number of DNA segments. In the second step, methylated (M) and unmethylated (U) primers are used which selectively amplify methylated and un-methylated DNA, and the amplicons are subsequently quantified with a standard curve using real-time PCR and two independent fluorophores to detect methylated/unmethylated DNA of each gene in the same well. Methylation is reported on a continuous scale.

The assay is easily performed on fresh or fixed cytological samples including brain and neuronal tissues, and cerebrospinal fluid.

### Primer and Probe Design for QM-MSP.

In practice of the methods of the present invention, quantitative real-time PCR methodology is adapted to perform quantitative methylation-specific PCR (QM-MSP) by utilizing the external primers pairs in round one (multiplex) PCR and internal primer pairs in round two (real time MSP) PCR. Thus, each set of genes has one pair of external primers and two sets of three internal primers/probe (internal sets are specific for unmethylated or methylated DNA). The external primer pairs can co-amplify a cocktail of genes, each pair selectively hybridizing to a member of the panel of genes being investigated using the invention method. Primer pairs are designed to exclude CG dinucleotides, thereby rendering DNA amplification independent of the methylation status of the promoter DNA sequence. Therefore, methylated and unmethylated DNA sequences internal to the binding sites of the external primers are co-amplified for any given gene by a single set of external primers specific for that gene. The external primer pair for a gene being investigated is complementary to the sequences flanking the CpG regions that is to be queried in the second round of QM-MSP. For example, the sequences of external primers set forth in Table 1 above are used for multiplex PCR (first round PCR) of genes associated with primary breast cancer (Fackler M. J. et al, Int. J Cancer (2003) 107:970-975; Fackler M. J. et al. Cancer Res (2004) 64:4442-4452).

Internal PCR primers used for quantitative real-time PCR of methylated and unmethylated DNA sequences (round two QM-MSP) are designed to selectively hybridize to the first amplicon produced by the first round of PCR for one or more members of the panel of DNA sequences being investigated using the invention method and to detect the methylation status, i.e., whether methylated (M) or unmethylated (U), of the CpG regions in the first amplicons to which they bind. Thus for each member of the starting panel of promoter DNA sequences used in an invention assay, separate QM-MSP reactions are conducted to amplify the first amplicon produced in the first round of PCR using the respective methylation-specific primer pair and using the respective unmethylated-specific primer pair.

In round two of QM-MSP a single gene or a cocktail of two or more genes can be co-amplified using distinguishable fluorescence labeled probes. The probes used in the round two QM-MSP of the invention method are designed to selectively hybridize to a segment of the first amplicon lying between the binding sites of the respective methylation-status specific internal PCR primer pair. Polynucleotide probes suitable for use in real-time PCR include, but are not limited to, a bi-labeled oligonucleotide probe, such as a molecular beacon or a TaqMan™ probe, which include a fluorescent moiety and a quencher moiety. In a molecular beacon the fluorescence is generated due to hybridization of the probe, which displaces the quencher moiety from proximity of the fluorescent moiety due to disruption of a stem-loop structure of the bi-labeled oligonucleotide. Molecular beacons, such as Amplifluor™ or TriStar™ reagents and methods are commercially available (Stratagene: Intergen). In a TaqMan™ probe, the fluorescence is progressively generated due to progressive degradation of the probe by Taq DNA polymerase during rounds of amplification, which displaces the quencher moiety from the fluorescent moiety. Once amplification occurs, the probe is degraded by the 5′-3′ exonuclease activity of the Taq DNA polymerase, and the fluorescence can be detected, for example by means of a laser integrated in the sequence detector. The fluorescence intensity, therefore, is proportional to the amount of amplification product produced.

Using the QM-MSP approach, it is possible to compile gene panels that are designed to address specific questions, or to provide intermediate markers or endpoints for clinical protocols. For example, when using de-methylating agents, a panel can be designed to query pathway-specific genes for their use as intermediate markers in specific trials of chemopreventive agents (Fackler M. J. et al. J Mammary Gland Biol Neoplasia (2003) 8:75-89).

Any of the known methods for conducting cumulative or quantitative “real time PCR” may be used in the second amplification step so long as the first amplicons in the first amplification product are contacted with one or more members of a set of polynucleotide probes that are labeled with distinguishable optically detectable labels, one or more members of the set being designed to selectively hybridize to one or more of the DNA sequences being tested, while the set cumulatively binds to the various DNA segments being tested contained in the first amplicons of the first amplification product. In addition, the first amplicons may also be contacted with such a set of probes and one or more members of a set of DNA sequence-specific methylation status-dependent inner primer pairs, wherein the set of inner primer pairs collectively bind to the various first amplicons in the first amplification product. In round two QM-MSP, additional genes can be co-amplified provided that each gene primer set incorporates a different color fluorescent probe.

For example, using the Applied Biosystem's 7500 Real-time PCR System, sample values are extrapolated from the standard curve for target and reference DNAs. This is called absolute quantitation.

QM-MSP: Percent Methylation (% M)=[copies Methylated TARGET gene/copies Methylated TARGET gene+copies un-methylated TARGET gene) copies] [100]; CMI=the sum of all % M values within the panel.

The phrase “a comparable normal DNA sample” as used herein means that the plurality of genomic DNA sequences that is being tested for methylation status, such as in a mammal, is matched with a panel of genomic DNA sequences of the same genes from a “normal” organism of the same species, family, and the like, for comparison purposes. For example, a substantial cumulative increase or decrease in the methylation level in the test sample as compared with the normal/benign sample (e.g., the cumulative incidence of the tumor marker in a test DNA panel compared with that cumulatively found in comparable apparently normal sample) is a reliable indicator of the presence of the condition being assayed.

### Use of Tailed Amplicon Multiplexed Methylation Specific PCR Methods

### PCR Amplification Step 1

In the first TAM-MSP PCR-amplification reaction, at least 2 or more, including 3, 4, 5 up to 10 DNA regions of interest are amplified simultaneously in a single multiplex PCR reaction. This reaction uses two or more sets of primers. Each primer in the set has a synthetic DNA sequence (or tail) added to the 5′ end. The same synthetic DNA tail is attached to the 5′end of each of the two or more forward and reverse primers used in this reaction. One or more of the primer sets have sequences that are specific for amplifying methylated DNA while the third primer set (the control) does not encompass any CG residues in the DNA, in order to determine total input of DNA going into the assay.

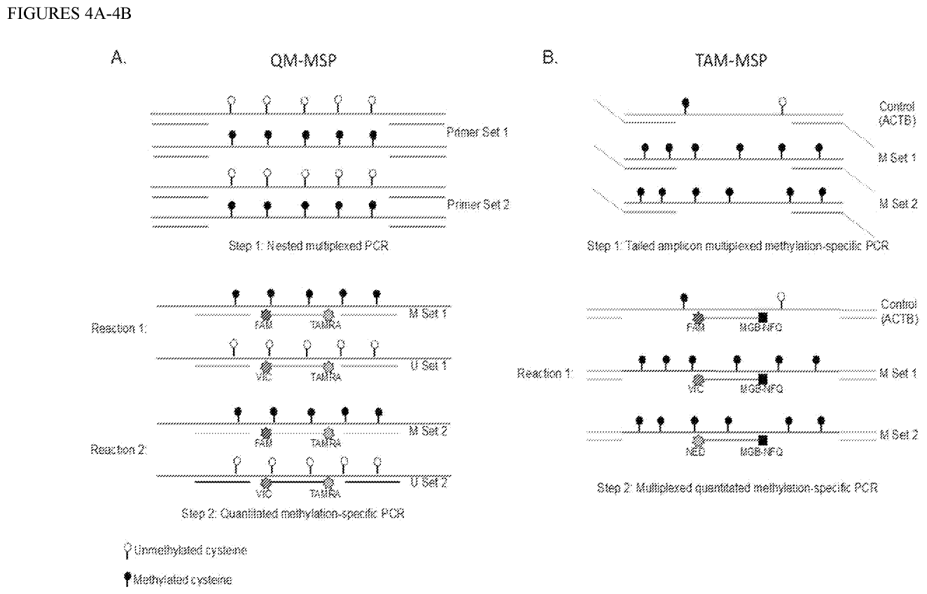

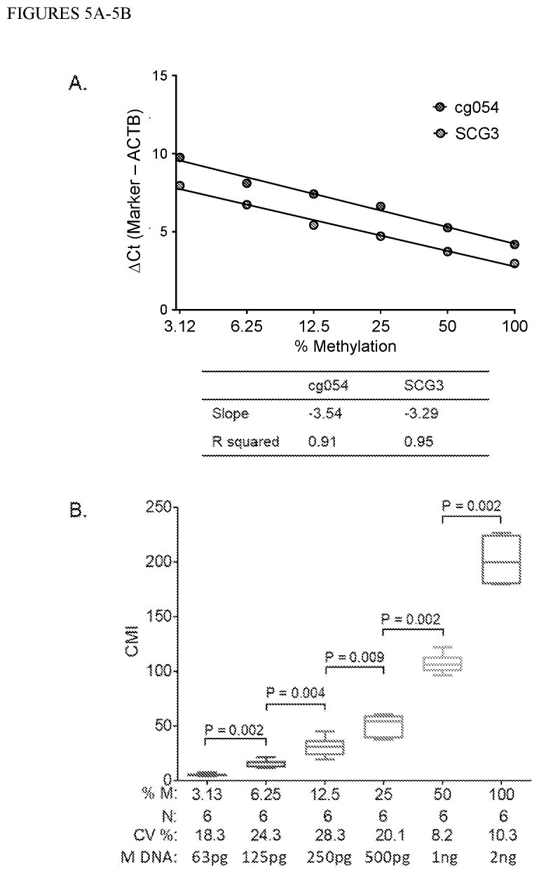

The tail sequences can vary, however the sequences should be distinct from typically methylated regions of DNA having C and G nucleotides. The sequences are between about 10 nucleotides to about 30 nucleotides, and can include 11, 12, 13, 14, 15, 20, 25 up to 30 nucleotides. In some embodiments, the tail has 18 nucleotides. The sequences can have a GC content between about 35% to about 50%, and in some embodiments, a GC content of about 45%. The tail sequences should not include any self-annealing or hairpin formations, and such information can be found in public databases such as the website (bioinformatics.org/sms2/pcr_primer_stats.html). In addition, the tail sequences should not bind to human genomic DNA. This can be found out using a variety of know methods including, for example, by testing them with an in-silico PCR website, such as the UC Santa Cruz PCR website (genome.ucsc.edu/cgi-bin/hgPcr) see FIG. 4.

TABLE 1
First PCT reaction TAM-MSP primer and probe characteristics

| | Primer/ | | Array | Primer Location | Size |
|---|---|---|---|---|---|
| Type | Probe Name | 5′ to 3′ Sequence | Target ID | (hg38) | (bp) |
| External | cg054_Ext_ | CATCTGCCTGAGTATGAACGAAGAGATAGC | cg05491001 | chr4:61,200, | 79 |
| | Tail_FM | GGCGG | | 782-61,200,860 | |
| External | cg054_Ext_ | CTTGATGTCAGTTAGGACGACCGAACCGAA | | | |
| | Tail_RM | CAACTA | | | |
| External | SCG3_Ext_ | CATCTGCCTGAGTATGAATGTTGGAGTTAT | cg01908954 | chr15:51,681, | 106 |
| | Tail_FM | TGATGTTATT | | 379-51,681,484 | |
| External | SCG3_Ext_ | CTTGATGTCAGTTAGGACCAAAACATAACT | | | |
| | Tail_RM | TTACTCTCACTCC | | | |
| External | ACTB_Ext_ | CATCTGCCTGAGTATGAAGTTTTTATTTAT | | Chr7:5,528, | 78 |
| | Tail_F | TTAGGAAGGAAG | | 269-5,528.346 | |
| External | ACTB_Ext_ | CTTGATGTCAGTTAGGACAAATCATCACCA | | | |
| | Tail_R | TTAACAATAAAC | | | |

### TAM-MSP Amplification Step 2

In the second PCR amplification reaction, all of the DNA regions of interest are amplified and measured in a single multiplex PCR reaction. A single set of primers that is specific for the synthetic tails, common for all three genes is used to amplify the two or more amplicons simultaneously. Different fluorescence markers (such as FAM, VIC, and NED, for example) are used in the reaction. Each fluorescence marker specific to each of the amplicons is used to quantitatively measure the number of amplicons in the reaction (see FIG. 4).

TABLE 2
Second PCT reaction TAM-MSP primer and probe characteristics

| | Primer/ | | Array | Primer Location | Size |
|---|---|---|---|---|---|
| Type | Probe Name | 5′ to 3′ Sequence | Target ID | (hg38) | (bp) |
| Target | cg054 M Probe | CGACCCCGAACTCTACGC | | | |
| Target | SCG3 M Probe | AACGCATACTCCATACACGC | | | |
| Target | ACTB Probe | CTACCCTAAAACACTCTTCC | | | |
| Target | Tail_Internal_F | CATCTGCCTGAGTATGAA | | | |
| Target | Tail_Internal_R | CTTGATGTCAGTTAGGAC | | | |

Ct values for each reaction are obtained using methylation specific PCR methods and apparatus known in the art, including, for example, the Applied Biosystems™ 7500 Fast Real-Time PCR software for methylated targets and the ACTB reference control (Ct=the cycle threshold at which signal fluorescence exceeds background). Thus, larger the number of amplicons of a certain gene in the reaction, the lower the cycle threshold. The reactions go for at least 30 to 50 cycles, preferably about 40 cycles.

For calculating % methylation, the ΔCt (Ct marker-Ct ACTB) value of each target marker was interpolated to the standard curves. The standard curves were generated using mixtures of fully methylated DNA and unmethylated DNA, to yield a range from 3% to 100% methylation at each dilution. The source of methylated DNA was SssI- (New England Biolabs, M0226S) treated human sperm DNA (HSD), and the source of unmethylated DNA was untreated HSD. The values of % methylation obtained from the samples of the subject are then compared to the % methylation obtained from normal or benign tissues of the same type. A % methylation level in the sample of the subject greater than the methylation level in from normal or benign tissues of the same type indicates hypermethylation of the gene of interest.

### Use of Quantitative Multiplex Methylation Specific PCR Method-cMethDNA Methods

In accordance with some embodiments, the inventive methods can also employ the QM-MSP variant method, cMethDNA to measure hypermethylation in a sample. The methods are disclosed in U.S. Pat. No. 10,450,609 and U.S. patent application Ser. No. 16/601,269, which are also incorporated herein as if set forth in their entireties. Briefly, the cMethDNA methods are a modification of the QM-MSP method specifically intended to quantitatively detect tumor DNA (or other circulating DNAs) in fluids such as serum or plasma at the lowest concentration (50 copies in 300 μl serum) yet reported. Unique compared to any other PCR-based assay, a small number of copies of a synthetic polynucleotide standard (the STDgene) is added to an aliquot of patient serum. In a standard procedure, a cocktail of standards for a plurality of genes of interest (TARGETgenes) is added to a sample of serum. Once total DNA is purified and processed, a PCR (pre-amplification, multiplex step) is performed wherein the STDgene and TARGETgene are co-amplified with the same external primer set in a manner independent of the methylation status of the TARGETgene. In the second step of nested PCR, amplicons present in a dilution of the first PCR reaction are subjected to real time PCR, and quantified for each gene in one or two well(s) by two-color real-time PCR. Products are calculated by absolute quantitation with internal primer sets specific for the methylated TARGETgene and associated STDgene. The methylation index of each gene (MI), as well as the cumulative methylation (CMI) of the gene panel is then determined based on copy number.

### Processing of Plasma and CSF DNA for TAM-MSP Assay

Whole blood is collected in a BD Vacutainer tube (BD Diagnostics, Franklin Lakes, NJ; catalog #367842). Plasma is separated with 2 consecutive centrifugations at 4500 RPM for 15 min at room temperature) according to manufacturer's directions within 1 week of collection. Plasma samples are stored at −80° C. as 2 ml aliquots and thawed on ice before using.

Cerebrospinal fluid (CSF) is collected into sterile collection tubes and was processed within 4 hours of collection. The CSF was isolated with 2 consecutive centrifugations for 10 min (1,500×g) at 4° C. The supernatant is transferred to a clean tube and stored at −80° C. as 1 ml aliquots and thawed on ice before using.

Plasma and CSF, as well as other fluids such as blood serum or saliva, is purified using the Quick-cfDNA serum & plasma kit (Zymo Research catalog #D4076), essentially according to the manufacturers' protocol although modified to accommodate 1 ml of plasma or CSF. For each new kit, stocks are prepared of Proteinase K (Invitrogen catalog #25530-049). The protocol given below is followed:

1. Pipet 250 μl S&P 5× Digestion Buffer and 100 μl Proteinase K to 1 ml of plasma, mix thoroughly and incubate at 55° C. for 30 min.

2. Add 2.7 ml of S&P DNA Binding Buffer to the digested mixture.

3. Transfer all of the mixture into the Zymo-Spin III-S column in a 50 ml conical tube and centrifuge at 1,000×g for 2 min.

4. Discard the top reservoir and place the Zymo-Spin III-S column in a collection tube.

5. Add 400 μl of S&P DNA Prep Buffer and centrifuge at 14,000×g for 30 sec. Discard the flow-through.

6. Add 700 μl S&P DNA Wash Buffer to the Zymo-Spin III-S column and centrifuge at 14,000×g for 30 sec. Discard the flow-through.

7. Add 400 μl S&P DNA Wash Buffer to the Zymo-Spin III-S column and centrifuge at 14,000×g for 1 min. Discard the flow-through.

8. Transfer the column into a new collection tube and add 70° C. 50 μl of H20 to the membrane and incubate for 3 min. Centrifuge at 14,000×g for 1 min and add the flow-through back to the column and incubate for 3 min. Centrifuge at 14,000×g for 1 min and transfer the contents to a 500 μl microcentrifuge tube for sodium bisulfite treatment. Keep on ice.

### Sodium Bisulfite-Mediated Conversion of Plasma and CSF DNA

1. Add 7.5 μl M-dilution buffer (Zymo Research catalog #D5002-2) to the DNA sample (represents all the DNA recovered from the purification step; final volume is ˜50 μl). Incubate at 42° C. for 20 minutes. Briefly centrifuge.

2. Prepare the CT Conversion Reagent (for each sample: water=71.4 μl, M-dilution buffer=17.6 μl, CT granular conversion reagent (Zymo Research catalog #D5001-1)=54 mg). Alternatively, use a ready-made vial per suitable for 11 plasma DNA samples: add 750 μl ddH2O+185 μl M-dilution buffer to the 1.7 ml brown vial containing 567 mg of CT reagent. This is enough for 11 plasma DNA samples. Rotate the solution in the dark at room temp for 10 min to dissolve. Use the reagent within 10 min.

3. Mix together 97.5 μl of CT Conversion Reagent with the sample thoroughly. Cap the tube, vortex briefly (or invert), centrifuge briefly. The final volume is ˜150 μl. Incubate the DNA solution in a PCR machine with a hot lid cycling 16 cycles of (95° C. 30 sec, 50° C. 1 hr). Hold at 4° C.

4: To perform DNA cleanup, place 600 μl of M-Binding Buffer (Zymo Research catalog #D5002-3) into a Zymo Spin Column IC (Zymo Research catalog #D5002-2) in a collection tube. Add the sample. Gently pipet up and down 10 times to mix the DNA with the binding buffer. Centrifuge at 13000 rpm for 30 seconds and change to a new collection tube. Discard the collection tube containing the flow through.

5. Add 100 μl of M-Wash Buffer (Zymo Research catalog #D5007-4) to the column and centrifuge at 13000 rpm for 30 seconds.

6. Add 200 μl M-Desulfonation Buffer (Zymo Research catalog #D5002-5) to the column and let the mixture sit at room temperature for 20 minutes. Centrifuge at 13000 rpm for 30 seconds. During this time, warm an aliquot of molecular grade water to 70° C.

7. Wash the column with 200 μl of M-Wash Buffer (Zymo Research catalog #D5007-4), invert the tube and centrifuge at 13000 rpm for 30 seconds. Discard the flow through. Change collection tubes. Repeat once, centrifuging a full minute.

8. Transfer the column to a new collection tube. Add 15 μl of the pre-heated water right to the top of the resin in the column. Allow the column to sit for 5-10 minutes at room temperature. Centrifuge the column at 13000 rpm for 1 minute to recover the DNA. Chill the sample on ice and then perform the TAM-MSP multiplex PCR assay using the entire sample.

### General Conditions

It should be recognized that an amplification “primer pair” as the term is used herein requires what are commonly referred to as a forward primer and a reverse primer, which are selected using methods that are well known and routine and as described herein such that an amplification product can be generated therefrom.

As used herein, the phrase “conditions that allow generation of an amplification product” or of “conditions that allow generation of a linear amplification product” means that a sample in which the amplification reaction is being performed contains the necessary components for the amplification reaction to occur. Examples of such conditions are provided herein and include, for example, appropriate buffer capacity and pH, salt concentration, metal ion concentration if necessary for the particular polymerase, appropriate temperatures that allow for selective hybridization of the primer or primer pair to the template nucleic acid molecule, as well as appropriate cycling of temperatures that permit polymerase activity and melting of a primer or primer extension or amplification product from the template or, where relevant, from forming a secondary structure such as a stem-loop structure. Such conditions and methods for selecting such conditions are routine and well known in the art (see, for example, Innis et al., “PCR Strategies” (Academic Press 1995); Ausubel et al., “Short Protocols in Molecular Biology” 4th Edition (John Wiley and Sons, 1999); “A novel method for real time quantitative RT-PCR” Gibson U. E. et al. Genome Res (1996) 6:995-1001; “Real time quantitative PCR” Heid C. A. et al. Genome Res (1996) 6:986-994).

As used herein, the term “selective hybridization” or “selectively hybridize” refers to hybridization under moderately stringent or highly stringent conditions such that a nucleotide sequence associates with a selected nucleotide sequence but not with unrelated nucleotide sequences. Generally, an oligonucleotide useful as a probe or primer that selectively hybridizes to a selected nucleotide sequence is at least about 15 nucleotides in length, usually at least about 18 nucleotides, and particularly about 21 nucleotides in length or more in length. Conditions that allow for selective hybridization can be determined empirically, or can be estimated based, for example, on the relative GC: AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual” (Cold Spring Harbor Laboratory Press 1989)).

In various embodiments, methylation-specific PCR can be used to evaluate methylation status of the target DNA. MSP utilized primer and/or probe sets designed to be “methylated-specific” by including sequences complementing only unconverted 5-methylcytosines, or, on the converse, “unmethylated-specific”, complementing thymine's converted from unmethylated cytosines. Methylation is then determined by the ability of the specific primer to achieve amplification. This method is particularly effective for interrogating CpG islands in regions of high methylation density, because increased numbers of unconverted methylcytosines within the target to be amplified increase the specificity of the PCR. In certain embodiments placing the CpG pair at the 3′-end of the primer also improves the specificity.

In certain embodiments, methylation can be evaluated using a Methy Light method. The Methy Light method is based on MSP, but provides a quantitative analysis using quantitative PCR (see, e.g., Eades et al. (2000) Nucleic Acids Res., 28(8): E32. doi: 10.1093/nar/28.8.e32). Methylated-specific primers are used, and a methylated-specific fluorescence reporter probe is also used that anneals to the amplified region. In alternative fashion, the primers or probe can be designed without methylation specificity if discrimination is needed between the CpG pairs within the involved sequences. Quantitation can be made in reference to a methylated reference DNA. One modification to this protocol to increase the specificity of the PCR for successfully bisulphite-converted DNA (ConLight-MSP) uses an additional probe to bisulphite-unconverted DNA to quantify this non-specific amplification (see, e.g., Rand et al. (2002) Methods 27(2): 114-120).

In various embodiments, the Methy Light methods utilize TAQMAN® technology, which is based on the cleavage of a dual-labeled fluorogenic hybridization probe by the 5′ nuclease activity of Taq-polymerase during PCR amplification (Eads et al. (1999) Cancer Res., 59: 2302-2306; Livak et al. (1995) PCR Meth. Appl., 4: 357-362; Lee et al. (1993) Nucleic Acids Res., 21: 3761-3766; Fink et al. (1998) Nat. Med., 4: 1329-1333). The use of three different oligonucleotides in the TAQMAN® technology (forward and reverse PCR primers and the fluorogenic hybridization probe) offers the opportunity for several sequence detection strategies.

In various embodiments, the methods described herein can involve nested PCR reactions and the reagents (e.g., primers and probes) for such nested PCR reactions. For example, in certain embodiments, methylation is detected for one, two, three, four, five, or six genes (gene promoters). Since bisulfite conversion of a DNA changes cytosine resides to uracil, but leave 5-methyl cytosine residues unaffected, the forward and reverse strands of converted (bisulfite-converted) DNA are no longer complementary. Accordingly, it is possible to interrogate the forward and reverse strands independently (e.g., in a multiplex PCR reaction) to provide additional specificity and sensitivity to methylation detection. In such instances, assaying of a single target can involve a two-plex multiplex assay, while assaying of two, three, four, five, or six target genes can involve four-plex, six-plex, 8-plex, 10-plex, or 12-plex multiplex assays. In certain embodiments the assays can be divided into two multiplex reactions, e.g., to independently assay forward and reverse strands. However, it will be recognized that when split into multiple multiplex assays, the grouping of assays need not be by forward or reverse, but can simply include primer/probe sets that are most compatible for particular PCR reaction conditions.

As indicated above, DLBCL and/or PCNSL can be identified, and/or staged and/or a prognosis therefor determined by the detection/characterization of the methylation state on the forward and/or reverse strand of gene promoters whose methylation (or lack thereof) is associated with the disease. It will be recognized that methylation (forward strand and/or reverse strand) of one or more of the genes shown herein for DLBCL and/or PCNSL can be determined to identify, and/or stage, and/or provide a prognosis for the indicated cancer. In certain embodiments, methylation status of all of the genes shown for a particular cancer (forward and/or reverse strand) can be determined in a single multiplex PCR reaction.

The inventive methods can be used to assess the methylation status of multiple genes, using very small quantities of DNA. A cumulative score of hypermethylation among multiple genes better distinguishes normal or benign from malignant tumors in bodily fluid samples as compared to the value of individual gene methylation markers.

It should be recognized that an amplification “primer pair” as the term is used herein requires what are commonly referred to as a forward primer and a reverse primer, which are selected using methods that are well known and routine and as described herein such that an amplification product can be generated therefrom.

In accordance with another embodiment of the present invention, it will be understood that the term “biological sample” or “biological fluid” includes, but is not limited to, any quantity of a substance from a living or formerly living patient or mammal. Such substances include, but are not limited to, blood, serum, plasma, urine, cerebrospinal fluid, neuronal tissue, vitreous humor, cells, organs, tissues, bone, bone marrow, synovial tissue, chondrocytes, synovial macrophages, endothelial cells, and skin. In some embodiments, the sample can be a FFPE sample. In one preferred embodiment, the sample is cerebrospinal fluid from a subject suspected of having PCNSL.

### Treatment Regimens

Treatment of PCNSL has evolved over the last decades, but no uniform consensus on the optimal treatment regimen exists currently. Experts in the field agree that high-dose methotrexate (HD-MTX) is the backbone of multimodal therapy, including other chemotherapeutic agents with and without radiation.

The role of surgery in PCNSL is generally restricted to stereotactic biopsy due to widespread and diffusely infiltrative tumor growth. A surgical resection increases the risk of permanent neurologic deficits in a disease that often involves deep structures and is highly chemosensitive.

HD-MTX is effective in patients with CNS metastases from lymphoma or lymphoid leukemias, and when added to WBRT, it enhanced response and prolonged survival in PCNSL. High doses of MTX are possible with the concomitant use of leucovorin, which prevents bone marrow and systemic organ damage while limiting the rescue of lymphoma cells in the CNS because of its poor BBB penetration. With the introduction of HD-MTX in combination with WBRT, ORR remained high (71% to 94%) but outcomes improved, with a median OS of 30 to 60 months and 5-year survival rates of 30% to 50%. Most studies were single-arm, phase II trials, with the exception of one study which demonstrated that the addition of cytarabine to HD-MTX and WBRT improved ORR from 40% to 69% and prolonged progression-free survival (PFS) from 3 to 18 months, suggesting that polychemotherapy is more effective than single agent HD-MTX.

In some treatments whole-brain radiotherapy (WBRT) was used to treat newly diagnosed PCNSL.

Rituximab, a monoclonal antibody directed against the B-cell surface antigen CD20, dramatically improves response and clinical outcome in DLBCL and was incorporated into first-line PCNSL treatment regimens. Rituximab is a large protein, but it can be detected in the CSF at a low level after systemic administration in patients with PCNSL and at the tumor site where the BBB is disrupted. The IELSG32 trial randomly assigned patients with PCNSLs to receive HD-MTX and cytarabine with or without thiotepa and with or without rituximab first-line treatment followed by WBRT (45 Gy) or high-dose chemotherapy with stem-cell rescue (HDC-ASCT) as consolidation. The results of the first randomization demonstrated that the addition of rituximab to HD-MTX/cytarabine improved ORR (73% v 53%) and median PFS (20 v 6 months). Moreover, the addition of thiotepa to rituximab and HD-MTX/cytarabine (MATRix regimen) further improved ORR to 86%, and median PFS has not been reached.

Currently, HD-MTX (>3 g/m²) and rituximab should be part of any induction treatment. Regimens currently used for induction are rituximab, HD-MTX, vincristine, and procarbazine (R-MVP), rituximab, HD-MTX, and temozolomide (R-MT), MATRix, or R-MVBP, depending on geographic region and physician preference. No comparison study has been conducted thus far. The only comparison study compared HD-MTX and temozolomide with HD-MTX, vincristine, and procarbazine (MVP) in an elderly population (age ≥60 years) in a multicenter phase II trial. Toxicity profiles were similar between the groups. ORR was 82% in the MVP group and 71% in the HD-MTX and temozolomide group, and median OS was 31 and 14 months, respectively. Although these trends were not statistically significant, the results favor the MVP regimen. For consolidation, radiation (23.4 or 45 Gy), conventional chemotherapy (cytarabine, etoposide plus cytarabine), HDC-ASCT (in younger patients and patients with adequate organ function), or observation (in elderly patients or those unable to tolerate additional treatment) is used. In addition, age and response to induction therapy should be used to guide the choice of consolidation.

### Treatment for DLBCL

Systemic Diffuse Large B-cell Lymphoma (DLBCL) tends to grow quickly. Most often, the treatment is chemotherapy (chemo), usually with a regimen of 4 drugs known as CHOP (cyclophosphamide, doxorubicin, vincristine, and prednisone), plus the monoclonal antibody rituximab (Rituxan). This regimen, known as R-CHOP, is most often given in cycles 3 weeks apart. Because this regimen contains the drug doxorubicin, which can damage the heart, it may not be suitable for patients with heart problems, so other chemo regimens may be used instead.

### Stage I or II

For DLBCL that is only in 1 or 2 lymph node groups on the same side of the diaphragm (the thin muscle that separates the chest from the abdomen), R-CHOP is often given for 3 to 6 cycles, which might be followed by radiation therapy to the affected lymph node areas.

### Stage III or IV

In some embodiments, the subject will be given 6 cycles of R-CHOP as first-line treatment. After several cycles, imaging tests such as a PET/CT scan may be performed to see how well treatment is working. Subjects who have a higher risk of the lymphoma coming back later in the tissues around the brain and spinal cord may be treated with chemo injected into the spinal fluid (called intrathecal chemotherapy). Another option is to give high doses of methotrexate intravenously.

For younger patients with a higher risk of the lymphoma coming back based on the International Prognostic Index (IPI) score, high-dose chemo followed by a stem cell transplant might be an option. Most often, if a transplant is done, it is as part of the first treatment, it should be done in a clinical trial.

If the lymphoma respond completely with treatment or if it recurs (comes back) after treatment, another chemotherapy treatment regimen can be begun. Several different regimens can be used, and they may or may not include rituximab. If the lymphoma shrinks with this treatment, it might be followed by a stem cell transplant if possible, as it offers the best chance of curing the lymphoma. Stem cell transplants are not effective unless the lymphoma responds to chemotherapy.

CAR-T cell therapies or a monoclonal antibody that targets CD79b can be considered if two or more treatments have been tried.

DLBCL can be cured in about half of all subjects, but the stage of the disease and the IPI score can have a large effect on this. Subjects with lower stages have better survival rates, as do subjects with lower IPI scores.

In some embodiments, a kit with the materials for performing the methylation analysis of one or more samples is described herein. The kit can include the primers, reagents and buffers needed.

In addition, the kits may include instructional materials containing directions (i.e., protocols) for the practice of the methods of this invention. While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Methods. The HyperMethylated Outlier Detector program was used to identify markers in a TCGA dataset of DLBC (N=48) and glioblastoma multiforme and lower grade glioma (GBMLGG, N=656). Marker performance was tested in samples from the GEO database (PCNSL, N=95; 11 primary CNS tumor types, N=2,112). Five of the eight markers were tested in formalin-fixed paraffin-embedded (FFPE) sections of PCNSL (N=25) and eight other primary CNS tumor types (N=25) by the laboratory assay, QM-MSP. The performance of a new and simplified assay, Tailed Amplicon Multiplexed Methylation-Specific PCR (TAM-MSP) was compared to QM-MSP.

### Study Design

Two independent datasets were analyzed in this study. Marker discovery Sample dataset 1 was from TCGA (HumanMethylation 450k) comprised of lymphoid neoplasm diffuse large B-cell lymphoma (DLBCL, N=48), and glioblastoma multiforme and brain lower grade glioma (GBMLGG, N=656). These data sets were downloaded from the website firebrowse (firebrowse.org).

Sample dataset 2 consisted of HumanMethylation 450k data downloaded from the Gene Expression Omnibus database (ncbi.nlm.nih.gov/geo/) and was used to test the performance of the 8 markers identified in Sample dataset 1. This sample set was comprised of PCNSL (GSE92676, N=95) and 11 other primary brain tumors (GSE36278, GSE44684, GSE50774, GSE58218, GSE61044, GSE103659, GSE104210, GSE42882, GSE70787, and GSE85212, N=2,112).

Sample set 3, the Wuhan clinical sample set, consisted of FFPE sections of archival CNS tumors. These were used to validate 5 of the 8 identified markers which were amenable to our laboratory assay, QM-MSP (16, 17). The top two-marker panel was also tested using the new TAM-MSP assay. Sample set 3 consisted of FFPE tissues of 25 samples of PCNSL and 25 samples of eight different types of CNS tumors (glioblastoma, N=6; astrocytoma, N=4; ependymoma, N=2; germinoma, N=2; medulloblastoma, N=3; oligodendrocytoma, N=4; meningioma, N=3; fibroma, N=1) collected at the Renmin Hospital of Wuhan University, China (Table 3), with institutional IRB approval. With a sample size of N=25 in each group, and an accuracy of 100%, the accuracy was estimated to be within 11% percentage points (lower confidence bound=89%).

TABLE 3
Table 3: Patient Characteristics of Validation Sample Set 3

| | CNS tumors* | | | | | | | | | |
|---|---|---|---|---|---|---|---|---|---|---|
| | | | | | | | | Solitary | CNS | PCNS- |
| | Glio. | Astro. | Ependy. | Germin. | Medullo. | Oligodendro. | Mening. | Fibroma | (Total) | DLBCL |
| N= | 6 | 4 | 2 | 2 | 3 | 4 | 3 | 1 | 25 | 25 |

Age-

| | | | | | | | | | | |
|---|---|---|---|---|---|---|---|---|---|---|
| Median | 46 | 36 | 36 | 21 | 12 | 44 | 47 | 32 | 39 | 58 |
| Range | 17-53 | 35-41 | 3-68 | 9-32 | 6-26 | 38-50 | 45-66 | — | 3-68 | 35-71 |

Gender

| | | | | | | | | | | |
|---|---|---|---|---|---|---|---|---|---|---|
| Male | 4 | 2 | 2 | 2 | 1 | 2 | 2 | — | 15 | 11 |
| Female | 2 | 2 | — | — | 2 | 2 | 1 | 1 | 10 | 14 |

WHO grade

| | | | | | | | | | | |
|---|---|---|---|---|---|---|---|---|---|---|
| I | — | — | — | — | — | — | — | — | — | — |
| II | — | 1 | — | — | — | — | — | — | 1 | — |
| III | — | 3 | 2 | — | — | 4 | 3 | 1 | 13 | — |
| IV | 6 | — | — | — | 3 | — | — | — | 9 | — |

Publicly available datasets of methylome data (HumanMethylation 450k) of B-cell, T-cell, monocyte (GSE59250), buffy coat (GSE109914), and normal brain (GSE128601), were downloaded from the Gene Expression Omnibus repository and used to determine if the identified 8 PCNSL markers were specific.

### Marker Identification and Analysis

DNA methylation levels for the CpGs in the publicly accessible HumanMethylation 450k data are reported as β values, which is defined as the ratio of the methylated allele intensity to the overall intensity. We removed markers from our analysis if more than 5% of the data in Sample set 1 or Sample set 2 had missing β values. Our marker discovery program named the HyperMethylated Outlier Detector (GitHub-HyperMethylated_Outlier_Detector) was used to identify promising methylation markers that distinguish between PCNSL or DLBCL, and other CNS tumors. This program first selects all markers that have a β value greater than 0.20 in the DLBCL samples, lymphoid and primary of the CNS. The selected markers were then filtered out if more than 5% of the GBMLGG samples have a β value greater than 0.15. Finally, the markers were sorted by their odds ratio. A single signature was derived for each of the two-marker panels by adding the cumulative β values of the two markers.

We also ascertained the specificity of detection of DLBCL and/or PCNSL by the 8 markers by comparing β value of tumor samples in Sample set 2 and different normal hematopoietic cells. That is, whether the methylation β value in the PCNSL were significantly higher than the methylation β values were low in normal peripheral blood cells and brain tissues by Mann Whitney analysis.

Statistical analysis was performed with R software (version 3.6.0). A custom script and the R package pROC was used to make the receiver operator characteristic (ROC) plot and to calculate the area under the curve (AUC) and 95% confidence interval (CI) for each of the 8 markers and all 28 two-marker panel combinations, using cumulative β values, in the Sample dataset 2 (18). The R package pheatmap was used to generate the heatmap plots (19). The boxplots were made with the R function boxplot. Mann-Whitney P values were calculated with the R function wilcox test. Histogram plots were made by GraphPad Prism version 8.1.2.

Extraction of DNA from FFPE Tissues

DNA was extracted from a 6 micron, deparaffinized sections of formalin fixed paraffin-embedded (FFPE) tissue, incubated overnight at 56° C. in 100 μl TNES buffer (10 mmol/l Tris, 150 mmol/L NaCl, 2 mmol/L EDTA, 0.5% SDS and 100 μg proteinase k (Invitrogen), and heat inactivated at 90° C. for 10 min. The DNA was then treated with sodium bisulfite as described previously (20-22).

### QM-MSP

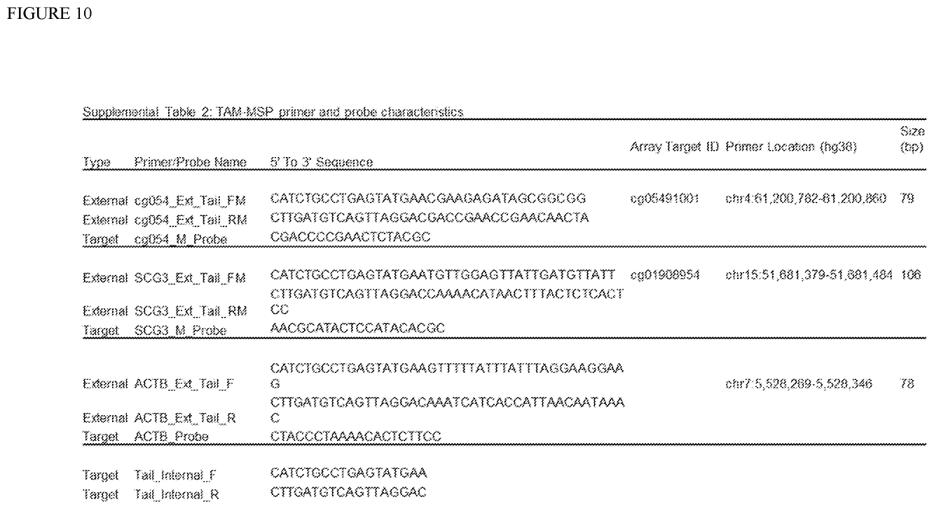

The QM-MSP assay was used to validate the selected markers in DNA extracted from FFPE sections of tumors in Sample set 3. The QM-MSP result for the panel of genes in each sample was expressed as Cumulative Methylation Index (CMI), the sum of percent methylation for each gene in the panel (16, 17). Sequences of QM-MSP primers and probes (ThermoFisher Scientific) are listed in FIG. 10. All methylated target probes were labeled with FAM and the unmethylated probes were labeled with VIC. Both probes used TAMRA as quencher.

### TAM-MSP

The TAM-MSP assay procedure also required two sequential PCR reactions. In the first PCR reaction (the tailed multiplexed step), 2 μl (of 100 μl lysate from a FFPE tissue section) of sodium bisulfite-treated DNA was added to 48 μl of reaction buffer [1.25 mM deoxynucleotide triphosphates, 16.6 mM (NH₄)₂SO₄, 67 mM Tris (pH 8.8), 6.7 mM MgCl₂, 10 mM β-mercaptoethanol, 0.1% DMSO, and 5 unit of Platinum Taq (Invitrogen) containing 400 nM each of the forward and reverse primers. Conditions of the qPCR were 95° C. for 5 min, followed by 35 cycles of 95° C. for 30 s, 56° C. for 45 s, and 72° C. for 45 s, with a final extension cycle of 72° C. for 7 min. The PCR products were diluted to 200 μl with reaction buffer and stored at −20° C.

For the second round (the qMSP step), 2 μl of the diluted PCR product from reaction 1 was further diluted 1:100. The diluted DNA was added to the qMSP reaction buffer containing 16.6 mM (NH₄)₂SO₄, 67.0 mM Tris (pH 8.8), 6.7 mM MgCl₂, 10.0 mM β-mercaptoethanol, 0.1% DMSO, 200 μM deoxynucleotide triphosphates, 1.25 units Ramp Taq (Thomas Scientific), 50 μg/ml tRNA (Invitrogen) and 300 nM ROX (Invitrogen) in a final volume of 20 μl. Seven hundred nM each of primers (forward and reverse) and 200 nM labeled probe (Applied Biosystems) were also present. The reaction was carried out in a 96-well reaction plate in an Applied Biosystems 7500 Fast Real-Time PCR (Applied Biosystems). The reaction conditions were as follows: 95° C. for 7 min, followed by 40 cycles of 95° C. for 15 s and 62° C. for 1 min.

To quantitatively assess three markers in a single reaction (ACTB, marker 1, marker 2) by TAM-MSP, FAM, VIC and NED were used as gene-specific fluorophores, while MGB-NFQ was used as the common quencher for all three probes. The sequences of the TAM-MSP primers and probes are listed in FIG. 11.

Ct values (Ct=the cycle threshold at which signal fluorescence exceeds background) were obtained using the Applied Biosystems™ 7500 Fast Real-Time PCR software for methylated targets and ACTB reference control. For calculating % methylation, the ΔCt (Ct marker−Ct ACTB) value of each target marker was interpolated from the standard curves of mixtures of fully methylated DNA [SssI (New England Biolabs, M0226S) treated human sperm DNA (HSD)], and unmethylated DNA (untreated HSD), with dilutions ranging from 100% to 3% methylation. Six replicate assays for each dilution of DNA were performed. This provided a standard curve that enabled us to determine, in test samples, the percent methylation in each gene and the cumulative methylation index (CMI), by adding percent methylation in each marker relative to b-actin in the 2-marker panel.

Interassay reproducibility for QM-MSP analysis was calculated from the 6 replicate assays using mixtures of fully methylated and unmethylated DNA used for preparing the standard curve as described above. Reproducibility was reported as Coefficient of Variation (CV) as calculated by GraphPad Prism version 8.1.2.

Inter-platform reproducibility was tested by analyzing the 25 CNS tumors and 25 PCNSL samples by both TAM-MSP and QM-MSP. The R function cor.test was used to calculate the spearman rho value and P value.

### Example 1

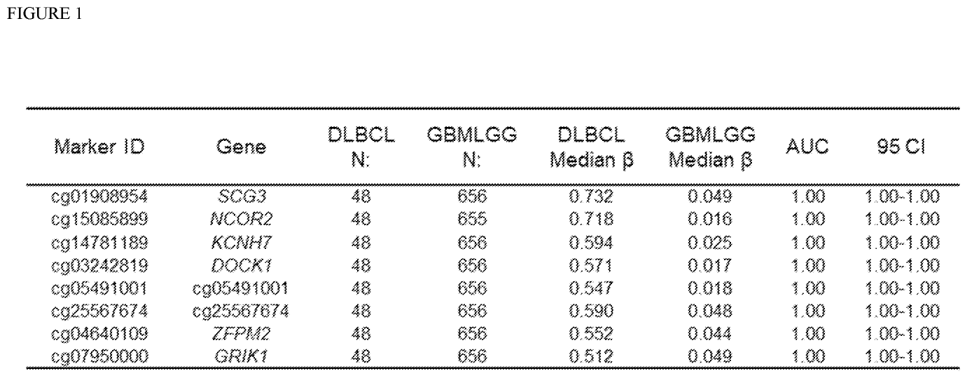

DNA methylation markers of DLBCL and/or PCNSL were identified by comparing TCGA datasets of 48 lymphoid neoplasm diffuse large B-cell lymphoma with datasets of glioblastoma multiformae and lower grade gliomas (GBMLGG, N=656) using our HyperMethylated Outlier algorithm. Through the use of this algorithm, we were able to identify 8 markers that were hypermethylated (β value greater than 0.20) in all of the PCNSL samples and had a β value less than 0.15 in all of the GBMLGG. These 8 markers, NCOR2, KCNH7, DOCK1, cg05491001, SCG3, ZFPM2, GRIK1 and cg25567674, were identified as DLBC-specific outliers, and had individual area under the ROC, of 1.00 (95% CI: 1.00-1.00) (FIG. 1).

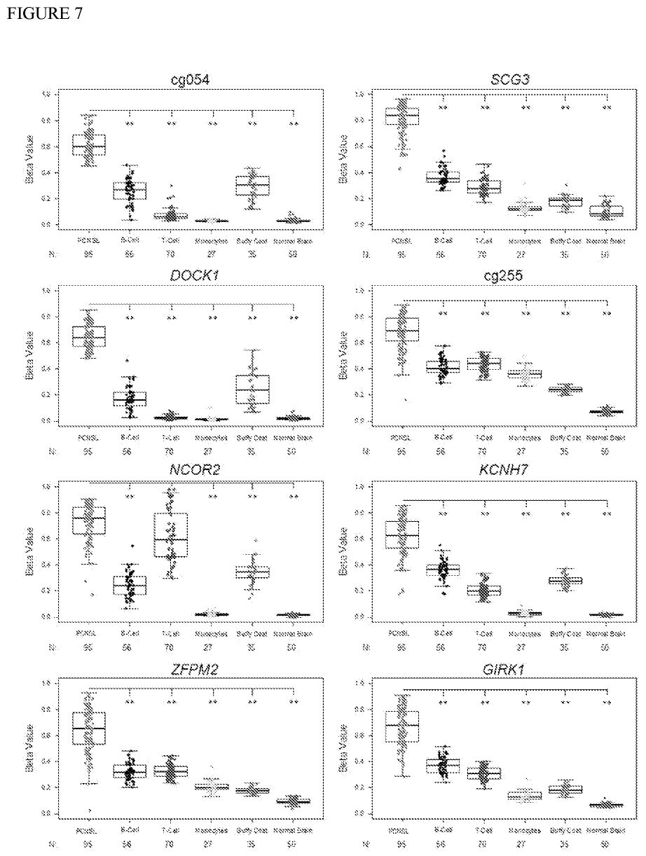

To rule out the possible contribution of methylation positivity by these markers in normal brain tissue or peripheral blood cell infiltration to signals generated in PCNSL, levels of methylation in the 8 markers were compared in an independent dataset of PCNSL (N=95), and normal B-cells (N=56), T-cells (N=70), monocytes (N=27), buffy coat (N=35), and normal brain (N=50). All 8 markers showed significantly higher levels of DNA methylation (P<0.001, Mann-Whitney) in the PCNSL tissues (median β values, range—0.601-0.838) compared to the normal B-cells (median β values, range-0.164-0.364) (FIG. 7). Furthermore, methylation levels in PCNSL for the 8 markers were significantly higher than normal T-cells (median β value, 0.232-0.592), monocytes (median β value, 0.013-0.197), buffy coat (median β value, 0.142-0.347), and normal brain tissue (median β value, 0.015-0.092) (FIG. 7). NCOR2 showed relatively high levels of methylation in normal T-cells (medium β value of 0.592). However, the median β value for NCOR2 (0.759) in PCNSL tissues was still significantly higher (P<0.001, Mann Whitney) than normal T-cells (FIG. 7).

### Example 2

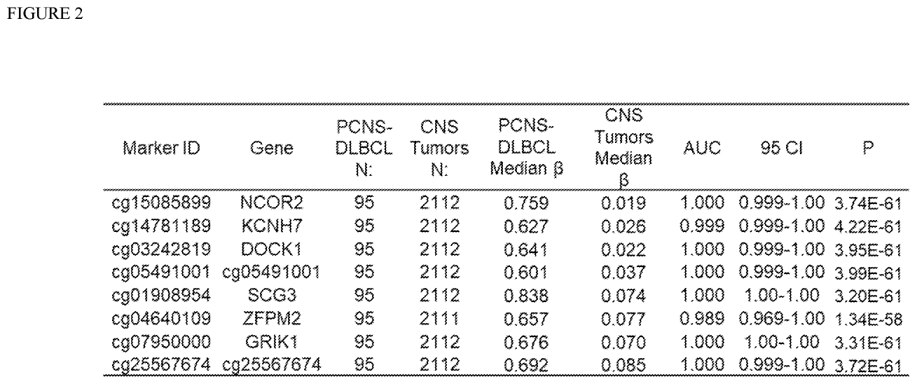

The 8 candidate markers were then tested in independent, publicly available data compiled by us from several Gene Expression Omnibus submissions. In addition to 95 PCNSL samples, these datasets include 2,112 CNS tumors representing 11 tumor types. In this test dataset, each of the 8 markers could accurately distinguish between the PCNSL samples and the 11 other CNS tumor types (AUC ranging between 0.989 and 1.00, P<0.001) (FIG. 2). The median β values of the 8 markers in the PCNSL ranged between 0.601-0.838 compared to the other CNS tumors where the median β values ranged between 0.019-0.085 (FIG. 2).

The above tests indicated that even one marker would be sufficient to distinguish between PCNSL and other CNS neoplasms. However, tumors can be heterogeneous. To account for such a possibility and to select the best performing marker set, cumulative methylation of two-markers in all 28 possible combinations in the Sample 2 dataset was tested. It was found that five of the 28 (18%) two-marker combinations achieve 100% accuracy in distinguishing PCNSL samples from the other 11 CNS tumor types. The top 5, two-marker panels were combinations of: SCG3 and DOCK1, SCG3 and cg054, SCG3 and cg255, DOCK1 and cg054, and GRIK1 and cg255.

### Example 3

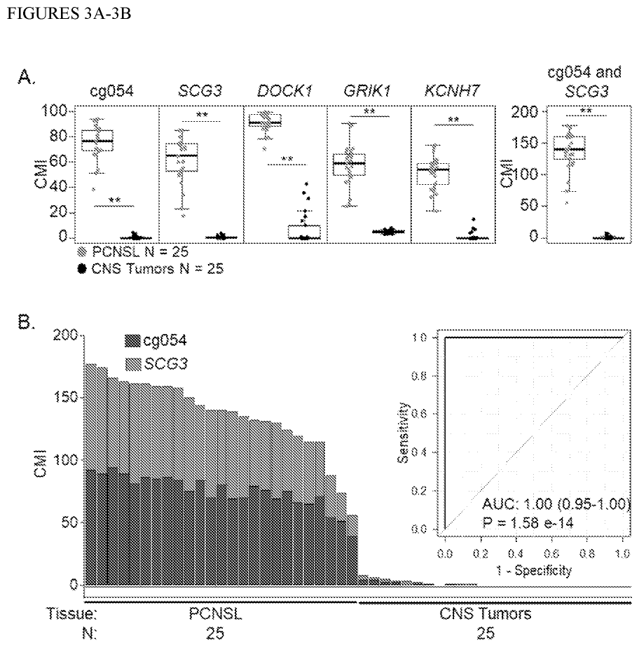

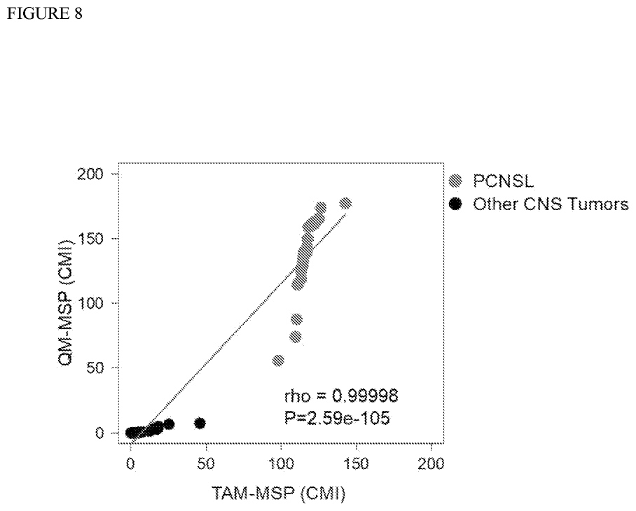

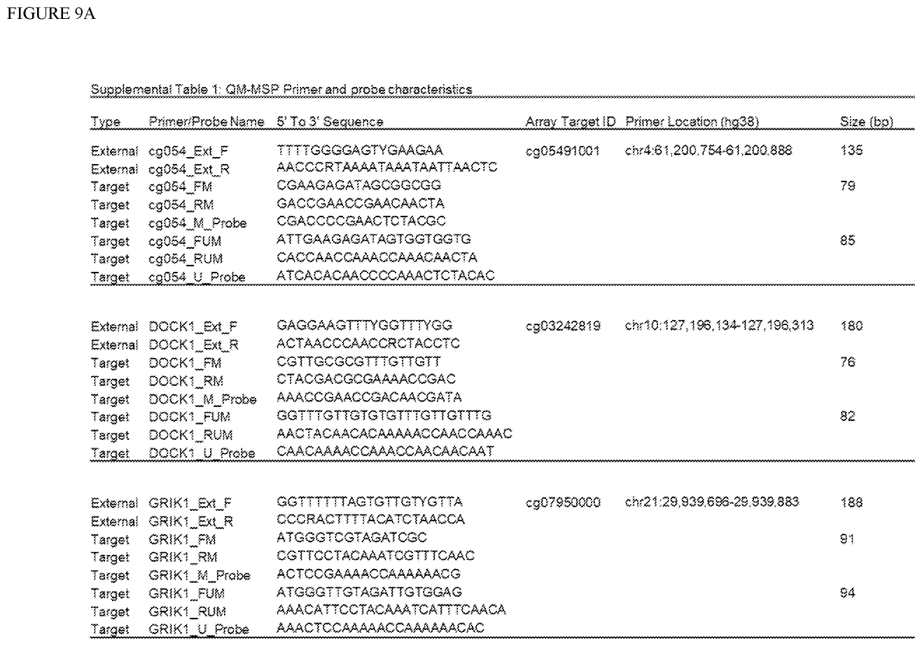

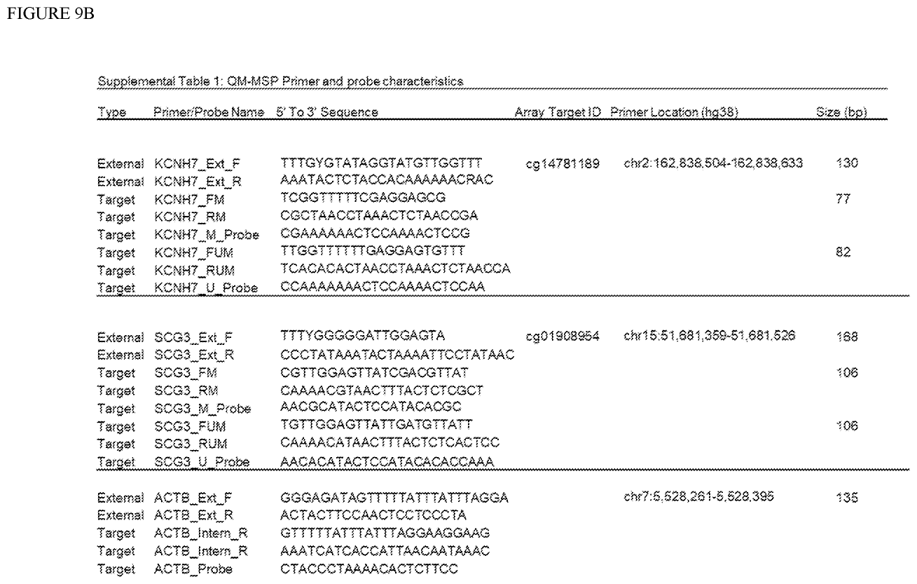

Next, to validate these findings using a quantitative, laboratory, methylation-specific PCR assay, QM-MSP (16, 17), we developed primers and Taqman probes successfully for 5 out of 8 markers. Three of the markers, cg255, NCOR2 and ZFPM2, were in genomic regions that were not amenable to designing efficient primers and probes. We found that the Ct values for the fully methylated and fully unmethylated DNA controls for SCG3 and cg054 amplified with the earliest Cts in comparison to the other 3 markers. Combining the two attributes of 100% accuracy in the GEO Sample dataset 2 and the high efficiency of amplification in the QM-MSP assay, SCG3 and cg054 were selected as the optimal two-marker panel for further investigation. In an independent sample set of FFPE tissues of PCNSL (N=25) and CNS tumors (N=25) comprised of 8 different tumor types, each of the 5 markers analyzed independently as well as the two-marker panel of distinguished between these two tissue types with 100% accuracy (AUC=1.00, 95% CI=1.00-1.00, P<0.001) (FIGS. 3A-C, FIG. 8).

### Example 4

The best performing two-marker panel consisted of SCG3 and cg054, and the pair was selected to develop a more simplified, quantitative methylation-specific PCR assay that could be conducted in a single triplexed qPCR reaction. In our novel TAM-MSP assay, ACTB was chosen as a DNA loading/reference control (FIG. 4B). All three sets of forward and reverse primers had synthetic 5′ tails which were incorporated into the PCR product in the first (multiplexing) reaction. In the second reaction, all three markers were amplified in the same well, with the three gene-specific sets of primers, along with probes, each tagged with a different fluorophore.

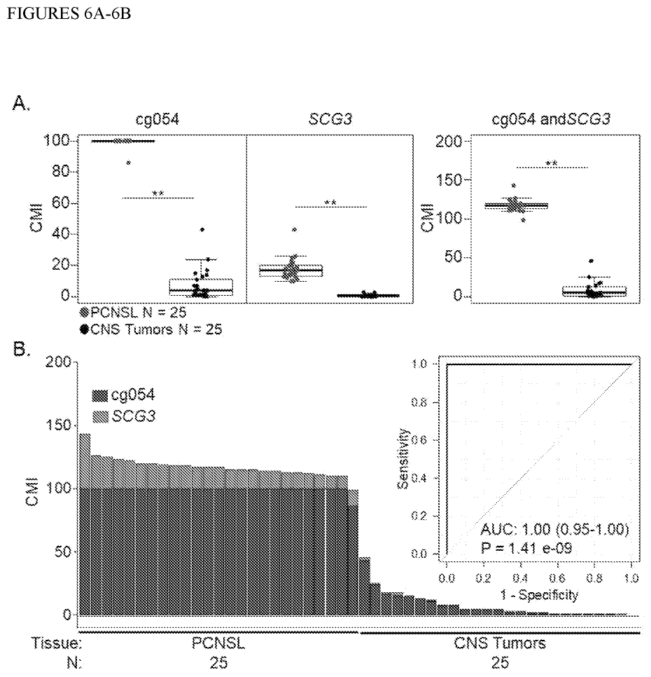

The novel TAM-MSP assay of the present invention showed linearity (R²>0.90) in DNA samples containing 100% to 3% methylation, and accurately measured as little as 62 pg of methylated DNA, an equivalent of 10 cells (FIGS. 5A and B). Both markers selected, SCG3 and cg054, distinguished between PCNSL and the eight other CNS tumors with 100% accuracy (AUC=1.00, 95% CI=1.00-1.00, P<0.001) (FIGS. 6A-C). The newly developed assay performed with equal efficiency as QM-MSP: the Spearman correlation between the cumulative methylation of the two-marker set by TAM-MSP and QM-MSP was near identical (rho-0.99998 P=2.59e-105) (FIG. 8).

The TAM-MSP method does include specific modifications that were designed with liquid biopsies in mind. Previous studies have shown that significant improvement in the sensitivity of the liquid biopsy assay can be achieved by increasing PCR efficiency to amplify the tumor DNA target (24). Several studies have shown that the size of ctDNA in plasma is approximately 165 bp (25-27). In TAM-MSP, by placing primers within the methylated region in the first amplification step, we achieved two goals. First, we increased the efficiency of the PCR reaction by amplifying just the regions of DNA that are informative for the assay, and second, the amplicon size was decreased to be within 150 bp, closer to the size of the DNA fragments found in circulation. With these new design features built into the TAM-MSP assay, we believe only minor modifications will be needed to measure methylation in DLBCL and/or PCNSL in circulating cfDNA in CSF and blood. CSF is likely to provide a low volume, high cfDNA level template for TAM-MSP, but is nevertheless invasive.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Batchelor T T, DeAngelis L M. Lymphoma and leukemia of the nervous system. New York: Springer; 2013.
2. Lim T, Kim S J, Kim K, Lee J I, Lim D H, Lee D J, Baek K K, Lee H Y, Han B, Uhm J E, Ko Y H, Kim W S. Primary CNS lymphoma other than DLBCL: a descriptive analysis of clinical features and treatment outcomes. Ann Hematol. 2011; 90(12): 1391-8. doi: 10.1007/s00277-011-1225-0. PubMed PMID: 21479535; PMCID: PMC3210363.
3. Ostrom Q T, Gittleman H, Fulop J, Liu M, Blanda R, Kromer C, Wolinsky Y, Kruchko C, Barnholtz-Sloan J S. CBTRUS Statistical Report: Primary Brain and Central Nervous System Tumors Diagnosed in the United States in 2008-2012. Neuro Oncol. 2015; 17 Suppl 4:iv1-iv62. doi: 10.1093/neuonc/nov 189. PubMed PMID: 26511214; PMCID: PMC4623240.
4. Ferreri A J, Cwynarski K, Pulczynski E, Ponzoni M, Deckert M, Politi L S, Torri V, Fox C P, Rosee P L, Schorb E, Ambrosetti A, Roth A, Hemmaway C, Ferrari A, Linton K M, Ruda R, Binder M, Pukrop T, Balzarotti M, Fabbri A, Johnson P, Gorlov J S, Hess G, Panse J, Pisani F, Tucci A, Stilgenbauer S, Hertenstein B, Keller U, Krause S W, Levis A, Schmoll H J, Cavalli F, Finke J, Reni M, Zucca E, Illerhaus G, International Extranodal Lymphoma Study G. Chemoimmunotherapy with methotrexate, cytarabine, thiotepa, and rituximab (MATRix regimen) in patients with primary CNS lymphoma: results of the first randomisation of the International Extranodal Lymphoma Study Group-32 (IELSG32) phase 2 trial. Lancet Haematol. 2016; 3(5):e217-27. doi: 10.1016/S2352-3026(16)00036-3. PubMed PMID: 27132696.
5. Chen C, Sun P, Cui J, Yan S, Chen H, Xia Y, Bi X, Liu P, Wang Y, Yang H, Nie M, Zhang X W, Jiang W, Li Z M. High-dose Methotrexate plus temozolomide with or without rituximab in patients with untreated primary central nervous system lymphoma: A retrospective study from China. Cancer Med. 2019; 8(4): 1359-67. doi: 10.1002/cam4.1906. PubMed PMID: 30821418; PMCID: PMC6488123.
6. Porter A B, Giannini C, Kaufmann T, Lucchinetti C F, Wu W, Decker P A, Atkinson J L, O'Neill B P. Primary central nervous system lymphoma can be histologically diagnosed after previous corticosteroid use: a pilot study to determine whether corticosteroids prevent the diagnosis of primary central nervous system lymphoma. Ann Neurol. 2008; 63(5):662-7. doi: 10.1002/ana.21366. PubMed PMID: 18481293.
7. Labak C M, Holdhoff M, Bettegowda C, Gallia G L, Lim M, Weingart J D, Mukherjee D. Surgical Resection for Primary Central Nervous System Lymphoma: A Systematic Review. World Neurosurg. 2019; 126:e1436-e48. doi: 10.1016/j.wneu.2019.02.252. PubMed PMID: 30904794.
8. Yang K, Wang S, Cheng Y, Tian Y, Hou J. Role of miRNA-21 in the diagnosis and prediction of treatment efficacy of primary central nervous system lymphoma. Oncol Lett. 2019; 17(3):3475-81. doi: 10.3892/ol.2019.9941. PubMed PMID: 30867786; PMCID: PMC6396200.
9. Baraniskin A, Deckert M, Schulte-Altedorneburg G, Schlegel U, Schroers R. Current strategies in the diagnosis of diffuse large B-cell lymphoma of the central nervous system. Br J Haematol. 2012; 156(4):421-32. doi: 10.1111/j. 1365-2141.2011.08928.x. PubMed PMID: 22077417.
10. Fontanilles M, Marguet F, Bohers E, Viailly P J, Dubois S, Bertrand P, Camus V, Mareschal S, Ruminy P, Maingonnat C, Lepretre S, Veresezan E L, Derrey S, Tilly H, Picquenot J M, Laquerriere A, Jardin F. Non-invasive detection of somatic mutations using next-generation sequencing in primary central nervous system lymphoma. Oncotarget. 2017; 8(29):48157-68. doi: 10.18632/oncotarget.18325. PubMed PMID: 28636991; PMCID: PMC5564634.
11. Hattori K, Sakata-Yanagimoto M, Suehara Y, Yokoyama Y, Kato T, Kurita N, Nishikii H, Obara N, Takano S, Ishikawa E, Matsumura A, Hasegawa Y, Chiba S. Clinical significance of disease-specific MYD88 mutations in circulating DNA in primary central nervous system lymphoma. Cancer Sci. 2018; 109(1):225-30. doi: 10.1111/cas.13450. PubMed PMID: 29151258; PMCID: PMC5765295.
12. Chen K, Ma Y, Ding T, Zhang X, Chen B, Guan M. Effectiveness of digital PCR for MYD88(L265P) detection in vitreous fluid for primary central nervous system lymphoma diagnosis. Exp Ther Med. 2020; 20(1):301-8. doi: 10.3892/etm.2020.8695. PubMed PMID: 32536998; PMCID: PMC7282034.
13. Downs B M, Mercado-Rodriguez C, Cimino-Mathews A, Chen C, Yuan J P, Van Den Berg E, Cope L M, Schmitt F, Tse G M, Ali S Z, Meir-Levi D, Sood R, Li J, Richardson A L, Mosunjac M B, Rizzo M, Tulac S, Kocmond K J, de Guzman T, Lai E W, Rhees B, Bates M, Wolff A C, Gabrielson E, Harvey S C, Umbricht C B, Visvanathan K, Fackler M J, Sukumar S. DNA Methylation Markers for Breast Cancer Detection in the Developing World. Clin Cancer Res. 2019; 25(21):6357-67. doi: 10.1158/1078-0432.CCR-18-3277. PubMed PMID: 31300453; PMCID: PMC6825533.
14. Fiano V, Trevisan M, Trevisan E, Senetta R, Castiglione A, Sacerdote C, Gillio-Tos A, De Marco L, Grasso C, Magistrello M, Tondat F, Ruda R, Cassoni P, Soffietti R, Merletti F. MGMT promoter methylation in plasma of glioma patients receiving temozolomide. J Neurooncol. 2014; 117(2): 347-57. doi: 10.1007/s11060-014-1395-4. PubMed PMID: 24519517.
15. Richter J, Ammerpohl O, Martin-Subero J I, Montesinos-Rongen M, Bibikova M, Wickham-Garcia E, Wiestler O D, Deckert M, Siebert R. Array-based DNA methylation profiling of primary lymphomas of the central nervous system. BMC Cancer. 2009; 9:455. doi: 10.1186/1471-2407-9-455. PubMed PMID: 20025734; PMCID: PMC2807878.
16. Gonzalez-Gomez P, Bello M J, Arjona D, Alonso M E, Lomas J, Aminoso C, de Campos J M, Sarasa J L, Gutierrez M, Rey J A. CpG island methylation of tumor-related genes in three primary central nervous system lymphomas in immunocompetent patients. Cancer Genet Cytogenet. 2003; 142(1):21-4. doi: 10.1016/s0165-4608(02)00799-9. PubMed PMID: 12660028.
17. Chu L C, Eberhart C G, Grossman S A, Herman J G. Epigenetic silencing of multiple genes in primary CNS lymphoma. Int J Cancer. 2006; 119(10):2487-91. doi: 10.1002/ijc.22124. PubMed PMID: 16858686.
18. Vogt J, Wagener R, Montesinos-Rongen M, Ammerpohl O, Paulus W, Deckert M, Siebert R. Array-based profiling of the lymphoma cell DNA methylome does not unequivocally distinguish primary lymphomas of the central nervous system from non-CNS diffuse large B-cell lymphomas. Genes Chromosomes Cancer. 2019; 58(1):66-9. doi: 10.1002/gcc.22687. PubMed PMID: 30284345.
19. Morton L M, Wang S S, Devesa S S, Hartge P, Weisenburger D D, Linet M S. Lymphoma incidence patterns by WHO subtype in the United States, 1992-2001. Blood. 2006; 107(1):265-76. doi: 10.1182/blood-2005-06-2508. PubMed PMID: 16150940; PMCID: PMC1895348.
20. Coiffier B, Lepage E, Briere J, Herbrecht R, Tilly H, Bouabdallah R, Morel P, Van Den Neste E, Salles G, Gaulard P, Reyes F, Lederlin P, Gisselbrecht C. CHOP chemotherapy plus rituximab compared with CHOP alone in elderly patients with diffuse large-B-cell lymphoma. N Engl J Med. 2002; 346(4):235-42. doi: 10.1056/NEJMoa011795. PubMed PMID: 11807147.
21. Larouche J F, Berger F, Chassagne-Clement C, Ffrench M, Callet-Bauchu E, Sebban C, Ghesquieres H, Broussais-Guillaumot F, Salles G, Coiffier B. Lymphoma recurrence 5 years or later following diffuse large B-cell lymphoma: clinical characteristics and outcome. J Clin Oncol. 2010; 28(12): 2094-100. doi: 10.1200/JCO.2009.24.5860. PubMed PMID: 20308668.
22. El-Galaly T C, Jakobsen L H, Hutchings M, de Nully Brown P, Nilsson-Ehle H, Szekely E, Mylam K J, Hjalmar V, Johnsen H E, Bogsted M, Jerkeman M. Routine Imaging for Diffuse Large B-Cell Lymphoma in First Complete Remission Does Not Improve Post-Treatment Survival: A Danish-Swedish Population-Based Study. J Clin Oncol. 2015; 33(34):3993-8. doi: 10.1200/JCO.2015.62.0229. PubMed PMID: 26438115.
23. Fackler M J, Lopez Bujanda Z, Umbricht C, Teo W W, Cho S, Zhang Z, Visvanathan K, Jeter S, Argani P, Wang C, Lyman J P, de Brot M, Ingle J N, Boughey J, McGuire K, King T A, Carey L A, Cope L, Wolff A C, Sukumar S. Novel methylated biomarkers and a robust assay to detect circulating tumor DNA in metastatic breast cancer. Cancer Res. 2014; 74(8): 2160-70. doi: 10.1158/0008-5472.CAN-13-3392. PubMed PMID: 24737128; PMCID: PMC4327879.
24. Fackler M J, Cho S, Cope L, Gabrielson E, Visvanathan K, Wilsbach K, Meir-Levi D, Lynch C F, Marks J, Geradts J, Regan M M, Viale G, Wolff A C, Sukumar S, Umbricht C B. DNA methylation markers predict recurrence-free interval in triple-negative breast cancer. NPJ Breast Cancer. 2020; 6:3. doi: 10.1038/s41523-020-0145-3. PubMed PMID: 32025567; PMCID: PMC6994477.
25. Fackler M J, Mcveigh M, Mehrotra J, Blum M A, Lange J, Lapides A, Garrett E, Argani P, Sukumar S. Quantitative multiplex methylation-specific PCR assay for the detection of promoter hypermethylation in multiple genes in breast cancer. Cancer research. 2004; 64(13):4442-52. doi: 10.1158/0008-5472.CAN-03-3341. PubMed PMID: 15231653.
26. Swift-Scanlan T, Vang R, Blackford A, Fackler M J, Sukumar S. Methylated genes in breast cancer: associations with clinical and histopathological features in a familial breast cancer cohort. Cancer biology & therapy. 2011; 11(10): 853-65. PubMed PMID: 21383541; PMCID: PMC3116929.
27. Robin X, Turck N, Hainard A, Tiberti N, Lisacek F, Sanchez J C, Muller M. pROC: an open-source package for R and S+ to analyze and compare ROC curves. Bmc Bioinformatics. 2011; 12:77. doi: 10.1186/1471-2105-12-77. PubMed PMID: 21414208; PMCID: PMC3068975.
28. Kolde R. Pheatmap: pretty heatmaps. R Package Version 61. 2012:1-7.
29. Fackler M J, Umbricht C B, Williams D, Argani P, Cruz L A, Merino V F, Teo W W, Zhang Z, Huang P, Visvananthan K, Marks J, Ethier S, Gray J W, Wolff A C, Cope L M, Sukumar S. Genome-wide methylation analysis identifies genes specific to breast cancer hormone receptor status and risk of recurrence. Cancer Res. 2011; 71(19):6195-207. doi: 10.1158/0008-5472.CAN-11-1630. PubMed PMID: 21825015; PMCID: PMC3308629.
30. Fackler M J, Mcveigh M, Evron E, Garrett E, Mehrotra J, Polyak K, Sukumar S, Argani P. DNA methylation of RASSFIA, HIN-1, RAR-beta, Cyclin D2 and Twist in in situ and invasive lobular breast carcinoma. Int J Cancer. 2003; 107(6):970-5. doi: 10.1002/ijc. 11508. PubMed PMID: 14601057.
31. Ladas I, Fitarelli-Kiehl M, Song C, Adalsteinsson V A, Parsons H A, Lin N U, Wagle N, Makrigiorgos G M. Multiplexed Elimination of Wild-Type DNA and High-Resolution Melting Prior to Targeted Resequencing of Liquid Biopsies. Clin Chem. 2017; 63(10): 1605-13. doi: 10.1373/clinchem.2017.272849. PubMed PMID: 28679646; PMCID: PMC5914173.
32. Mouliere F, Chandrananda D, Piskorz A M, Moore E K, Morris J, Ahlbom L B, Mair R, Goranova T, Marass F, Heider K, Wan J C M, Supernat A, Hudecova I, Gounaris I, Ros S, Jimenez-Linan M, Garcia-Corbacho J, Patel K, Ostrup O, Murphy S, Eldridge M D, Gale D, Stewart G D, Burge J, Cooper W N, van der Heijden M S, Massie C E, Watts C, Corrie P, Pacey S, Brindle K M, Baird R D, Mau-Sorensen M, Parkinson C A, Smith C G, Brenton J D, Rosenfeld N. Enhanced detection of circulating tumor DNA by fragment size analysis. Sci Transl Med. 2018; 10(466). doi: 10.1126/scitranslmed.aat4921. PubMed PMID: 30404863; PMCID: PMC6483061.

1. A method for identifying diffuse large B-Cell lymphoma (DLBCL) and/or Primary Central Nervous System (PCNSL) in a biological sample comprising detecting the presence of one or more hypermethylated CpG regions in one or more genes of interest in the DNA from the sample, wherein the one or more genes of interest are selected from the group consisting of: NCOR2, KCNH7, DOCK1, cg05491001, SCG3, ZFPM2, GRIK1 and cg25567674; and identifying the CpG regions of the one or more of the genes of interest as hypermethylated by comparing the methylation level of the CpG regions of the one or more of the genes of interest in the sample to the methylation level of the CpG regions of the one or more of the genes of interest in normal or benign tissue sample.

2. A method for detecting the presence of one or more hypermethylated CpG regions of one or more of the genes of interest in a biological sample from a subject comprising:
a) hybridizing nucleic acid obtained from the sample with one or more QM-MSP primers and probes specific for the CpG regions of the one or more genes of interest selected from the group consisting of: NCOR2, KCNH7, DOCK1, cg05491001, SCG3, ZFPM2, GRIK1 and cg25567674;
b) performing QM-MSP on the sample from a); and
c) detecting if any of the specific CpG regions of the one or more genes of interest of a) are hypermethylated compared to the level of methylation of the specific CpG regions of the one or more genes of interest in a normal or benign tissue sample.

3. A method for identifying DLBCL and/or PCNSL in a tissue from a subject comprising:
a) hybridizing nucleic acid obtained from the sample of the subject with one or more QM-MSP primers and probes specific for the CpG regions of the one or more genes of interest selected from the group consisting of: NCOR2, KCNH7, DOCK1, cg05491001, SCG3, ZFPM2, GRIK1 and cg25567674;
b) performing QM-MSP on the sample from a);
c) detecting if any of the specific CpG regions of the one or more genes of interest of a) are hypermethylated compared to the level of methylation of the specific CpG regions of the one or more genes of interest in a normal or benign tissue sample; and
d) identifying the subject as having DLBCL and/or PCNSL.

4-7. (canceled)

8. The method of claim 1, wherein at step c) detecting any of the specific CpG regions of one of the genes of interest of a) are hypermethylated compared to the level of methylation of the specific CpG regions of the one gene of interest in a normal or benign tissue sample.

9. The method of claim 1, wherein at step c) detecting any of the specific CpG regions of two of the genes of interest of a) are hypermethylated compared to the level of methylation of the specific CpG regions of the two genes of interest in a normal or benign tissue sample.

10. The method of claim 9, wherein the two gene regions of interest assayed are selected from the group consisting of: SCG3 and DOCK1, SCG3 and cg054, SCG3 and cg255, DOCK1 and cg054, and GRIK1 and cg255.

11. The method of claim 10, wherein the two gene regions of interest assayed are SCG3 and cg054.
